(19) United States
(12) Patent Application Publication
(10) Pub. No.: US 2024/0252711 A1
(43) Pub. Date: Aug. 1, 2024

(54) APPARATUS, METHOD AND SYSTEM FOR DISINFECTING AIR

(71) Applicants: Jonathan HIBBARD, Derby (GB); Stuart WEBB, Derby (GB); Glen John TYRRELL, Newborough (GB); Samuel WAKERLEY, Derby (GB); Jacob WAKERLEY, Derby (GB); James BROWBANK, Leicestershire (GB); Sebastian LEONTIES, Kent (GB); Joel BOND, Nottingham (GB)

(72) Inventors: Jonathan HIBBARD, Derby (GB); Stuart WEBB, Derby (GB); Glen John TYRRELL, Newborough (GB); Samuel WAKERLEY, Derby (GB); Jacob WAKERLEY, Derby (GB); James BROWBANK, Leicestershire (GB); Sebastian LEONTIES, Kent (GB); Joel BOND, Nottingham (GB)

(21) Appl. No.: 18/605,373

(22) Filed: Mar. 14, 2024

Related U.S. Application Data
(63) Continuation of application No. PCT/IB2022/058666, filed on Sep. 14, 2022.
(60) Provisional application No. 63/244,051, filed on Sep. 14, 2021.

(51) Int. Cl. A61L 9/20

(52) U.S. Cl. CPC A61L 9/20; A61L 2209/134; A61L 2209/14

(57) ABSTRACT

Disclosed herein are stages for a disinfection apparatus. Each stage may include one or more emitters configured to emit electromagnetic energy (e.g., ultraviolet light or infrared light or both) upon a fluid flow and/or one or more filters configured to filter the fluid flow. Also disclosed herein are disinfection apparatuses and methods for imparting electromagnetic energy upon a fluid flow. In some variations, the disinfection apparatus includes emitters configured to emit ultraviolet light or infrared light or both upon the fluid flow. A filter of the disinfection apparatus is configured to filter the fluid flow. A body of the disinfection apparatus defines a tortuous flow path delineating at least two turns such that the fluid flow changes direction at least twice as the fluid flow proceeds through the tortuous flow path.

### PRIORITY FIELD

This patent application claims the benefit of U.S. Provisional Patent Application No. 63/244,051, entitled “Disinfection Apparatus, Method, and System,” filed on Sep. 14, 2021, which is incorporated herein by reference in its entirety.

### TECHNICAL FIELD

The disclosure relates generally to fluid flow disinfection, and more specifically, to multi-stage disinfection through the use of ultraviolet light, infrared light and/or infrared heat, physical filtration and/or combinations thereof.

### BACKGROUND

Electromagnetic energy generated at specific wavelengths has both physical and chemical effects on structures. When generated at sufficient concentration or over sufficient time periods, these effects can adversely affect organic structures, including cells from micro-organisms. If sufficient energy is introduced to the surface of those structures, permanent and irrevocable damage may be experienced. This has been researched predominantly within the field of ultraviolet (UV) radiation.

Historically, UV radiation has been used repeatedly for sanitizing drinking water in an enclosed tube or vessel. Due to potential health risks, UV in hospitals is typically done via a wand or via a robotic device that administers dosing without the presence of an operator due to the danger of UV radiation on human eyes.

### BRIEF SUMMARY

The disclosure provides for disinfection apparatuses, stages for disinfection apparatuses, and methods for imparting electromagnetic energy upon a fluid flow. Each stage may include one or more emitters configured to emit electromagnetic energy (e.g., ultraviolet light or infrared light or both) upon a fluid flow and/or one or more filters configured to filter the fluid flow.

In a first aspect, the disclosure is directed to a disinfection apparatus. The apparatus includes a body defining at least one inlet and at least one outlet. One or more inlet fans are proximate the at least one inlet and configured to generate a fluid flow in a direction toward the at least one outlet. A first stage fluidly associated with the at least one inlet, the first stage defining a first tortuous flow path delineating at least two turns such that the fluid flow changes direction at least twice as the fluid flow proceeds through the first tortuous flow path, the first stage comprising one or more emitters configured to emit ultraviolet light at one or more wavelengths upon the fluid flow. A second stage fluidly associated with the first stage, the second stage comprising one or more emitters configured to emit infrared light at one or more wavelengths upon the fluid flow. A third stage fluidly associated with the second stage, the third stage comprising one or more metal filters constructed of one or more antibacterial metals and configured to filter the fluid flow. At least one outlet fluidly associated with the third stage.

In some variations, the at least one wavelength of the first stage is from 130 nm to about 405 nm upon the fluid flow. In some variations, the one or more emitters are configured to emit infrared light at a first wavelength of the at least one wavelength of the second stage is from 1200 nm to 1510 nm upon the fluid flow. In some variations, the one or more emitters are configured to emit infrared light at a second wavelength of the at least one wavelength of the second stage is from 1380 nm to 1800 nm upon the fluid flow. In some variations, at least one metal filter in the third stage is copper or copper alloy. In further variations, one or more outlet fans can be disposed between the first stage and the second stage and configured to force the fluid flow in a direction toward the at least one outlet. In some variations, the second stage defines a second tortuous flow path delineating at least two turns such that the fluid flow changes direction at least twice as the fluid flow proceeds through the second tortuous flow path. In further variations, the first tortuous flow path is defined by a plurality of spaced-apart walls, each of the plurality of spaced-apart walls spanning less than a full dimension of the first stage of the body such that the fluid flow proceeds around ends of each of the plurality of spaced-apart walls. In some variations, the first tortuous flow path is defined by a plurality of spaced-apart walls, each of the plurality of spaced-apart walls spanning a full dimension of the first stage of the body and defining an aperture configured to permit the fluid flow to pass therethrough. In some variations, the apertures are semicircular and configured to so as to create or increase turbulence and/or pressure of the fluid flow within the first stage.

In a second aspect, a stage for a disinfection apparatus may include one or more emitters. The one or more emitters may be configured to emit ultraviolet light upon a fluid flow.

In a third aspect, the one or more emitters may be configured to emit ultraviolet light at one or more wavelengths of about 130 nm to about 280 nm. In variations, the one or more emitters may be configured to emit ultraviolet light having a first wavelength peak at about 222 nm, a second wavelength peak at about 254 nm, and a third wavelength peak at about 265 nm. The one or more emitters may include a plurality of ultraviolet light emitting diodes. In the same or other variations, the one or more emitters may include at least one excimer lamp. In an aspect, the stage may further include one or more emitters configured to emit visible light at one or more wavelengths of about 380 nm to about 405 nm.

In a fourth aspect, another stage for a disinfection apparatus may include one or more emitters. The one or more emitters may be configured to emit infrared light upon a fluid flow. In an aspect, the one or more emitters may be configured to emit infrared light at one or more wavelengths of about 1300 nm to about 1600 nm.

In a fifth aspect, yet another stage for a disinfection apparatus may include one or more filters. The one or more filters may be configured to physically filter a fluid flow. In an aspect, the one or more filters may be configured to filter the fluid flow through inertial impaction, interception, dissemination, dispersion, and/or diffusion. The one or more filters may include at least one metal filter. In variations, the metal filter may be constructed of one or more antibacterial metals (e.g., at least one of copper, zinc, nickel, and combinations thereof).

In a sixth aspect, also provided herein is a disinfection apparatus. The disinfection apparatus may include one or more of the stages previously described. In an aspect, the disinfection apparatus may include a plurality of stages for imparting electromagnetic energy upon a fluid flow. The plurality of stages may include a first stage, a second stage, and/or a third stage. The first stage may include one or more emitters. The one or more emitters of the first stage may be configured to emit ultraviolet light upon the fluid flow. The second stage may include one or more emitters. The one or more emitters of the second stage may be configured to emit infrared light upon the fluid flow. The third stage may include one or more filters. The one or more filters of the third stage may be configured to filter the fluid flow through inertial impaction, interception, dissemination, dispersion, and/or diffusion.

In a seventh aspect, the disinfection apparatus may include a body defining a tortuous flow path. The tortuous flow path may delineate at least two turns such that the fluid flow changes direction at least twice as the fluid flow proceeds through the tortuous flow path. The tortuous flow path may be a substantially serpentine flow path.

In a eighth aspect, the disinfection apparatus may include one or more motors configured to force the fluid flow through the disinfection apparatus.

In an ninth aspect, the disinfection apparatus may further include a heating element configured to heat the fluid flow. In an aspect, the heating element may be configured to heat the fluid flow such that the fluid flow has a temperature of about 180° C. to about 300° C. as the fluid flow passes through the one or more filters.

In a tenth aspect, also provided herein is a disinfection apparatus. The disinfection apparatus may impart electromagnetic energy upon a fluid flow. The disinfection apparatus may include a body. The body may define a tortuous flow path. The tortuous flow path may delineate at least two turns such that the fluid flow changes direction at least twice as the fluid flow proceeds through the tortuous flow path. The disinfection apparatus may also include a plurality of emitters. The plurality of emitters may be within the body. The plurality of emitters may be configured to emit ultraviolet light or infrared light or both upon the fluid flow as the fluid flow proceeds through the tortuous flow path.

In an eleventh aspect, also provided herein is a method of imparting electromagnetic energy upon a fluid flow for disinfection thereof. The method may include flowing a fluid flow through a tortuous flow path. The tortuous flow path may delineate at least two turns such that the fluid flow changes direction at least twice as the fluid flow proceeds through the tortuous flow path. The method may further include emitting ultraviolet light or infrared light or both upon the fluid flow as the fluid flow proceeds through the tortuous flow path.

Additional aspects and features are set forth in part in the description that follows and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### DESCRIPTION OF THE DRAWINGS

The description will be more fully understood with reference to the following figures, which are presented as variations of the disclosure and should not be construed as a complete recitation of the scope of the disclosure, wherein:

FIG. 1A is a top view of a UV-C stage of a disinfection apparatus, according to an illustrative embodiment;

FIG. 1B is a front view of an arrangement of ultraviolet light emitting diodes, according to an illustrative embodiment;

FIGS. 1C-1H are illustrations of cone angles of an ultraviolet light emitting diode, according to an illustrative embodiment;

FIG. 2 is a top view of an IR stage of a disinfection apparatus, according to an illustrative embodiment;

FIG. 3A is a perspective view of a filtration stage of a disinfection apparatus, according to an illustrative embodiment;

FIG. 3B is a front view of a filtration device, according to an illustrative embodiment;

FIG. 3C is a side view of the filtration device of FIG. 3B;

FIG. 3D is a graph plotting pores per cm (PPcm) versus surface area multiplier, where the x-axis represents pores per cm (PPcm) and the y-axis represents the surface area multiplier, according to an illustrative embodiment;

FIG. 4 is a schematic illustration of a disinfection process, according to an illustrative embodiment;

FIG. 5A is a perspective view of a disinfection apparatus, according to an illustrative embodiment;

FIG. 5B is a top view of the disinfection apparatus of FIG. 5A;

FIG. 5B is a top view of the disinfection apparatus of FIG. 5A;

FIG. 5C is a side view of the disinfection apparatus of FIG. 5A;

FIG. 5D is an enlarged view of a portion of the disinfection apparatus of FIG. 5A;

FIG. 6 is a perspective view of a UV-C stage of the disinfection apparatus of FIG. 5A, according to an illustrative embodiment;

FIG. 7 is a graph plotting dosage versus fractional survival rate, where the x-axis represents time (in seconds) of a dosage rate of 222 nm, the y-axis represents the fractional survival rate (i.e., percentage), the solid circles represent 229E particles, and the empty circles represent OC43 particles, according to an illustrative embodiment; and

FIG. 8 is a graph plotting wavelength versus relative intensity to show the germicidal effectiveness of emitted light, where the x-axis represents various wavelengths (in nm), the y-axis represents relative intensity, and the dashed line shows that germicidal effects may occur at specific wavelengths, according to an illustrative embodiment.

FIG. 9 is an isometric view of a body of a disinfection apparatus, according to an illustrative embodiment.

FIG. 10A is an isometric view of a disinfection apparatus having a UV-C stage, an IR stage, and a filtration stage, according to an illustrative embodiment.

FIG. 10B is a front view of the disinfection apparatus of FIG. 10A, according to an illustrative embodiment.

FIG. 10C is a top view of the disinfection apparatus of FIG. 10A, according to an illustrative embodiment.

### DETAILED DESCRIPTION

The disinfection apparatuses and methods of use will be understood, both as to structure and operation, from the accompanying drawings, taken in conjunction with the accompanying description. It is noted that, for purposes of illustrative clarity, certain elements in various drawings may not be drawn to scale. Several variations of the apparatus are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

For purposes of this description, the terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

For purposes of this description, numerical values should be understood to include numerical values which are the same when reduced to the same number of significant figures and numerical values which differ from the stated value by less than the experimental error of conventional measurement technique of the type described in the present application to determine the value. Where a range of numerical values is provided, such ranges are inclusive of the recited endpoint and independently combinable. For example, the range of “from 20 nm to 100 nm” is inclusive of the endpoints, 20 nm and 100 nm, and all the intermediate values. The endpoints of the ranges and any values disclosed herein are not limited to the precise range or value; they are sufficiently imprecise to include values approximating these ranges and/or values.

For purposes of this description, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range of “about 20 to about 100” also discloses the range “from 20 to 100.”

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term.

Ultraviolet germicidal irradiation (UVGI) is a disinfection method that uses short-wavelength ultraviolet radiation (UVR) light to kill or inactivate or denature microorganisms. Short-wavelength (UV-C) can deactivate microorganisms by bringing about conformational change(s) to the polymer structures of ribonucleic acids (RNA) and deoxyribonucleic acids (DNA). Any irreparable changes to these acids can result in insufficient/unachievable RNA/DNA replication, rendering the microorganism inactive. It should be noted, some microorganisms do not require DNA to replicate and exist with RNA alone. UV-C will still sufficiently denature these microorganisms. RNA (ribonucleic acid) and DNA (deoxyribonucleic acid) are chemical compounds that can be made by the body. RNA (ribonucleic acid) and DNA (deoxyribonucleic acid) are made of chemicals called nucleotides. By damaging the chemical coding and removing vital elements of the cell structure, it leaves them unable to perform vital cellular functions.

Without wishing to be limited to a particular mechanism or mode of action, generation of radical species using oxide compounds present in cells can be further achieved by the use of specifically tuned UV-C wavelength. UV-C wavelengths cause formation of radical oxygen species. In some variations, these wavelengths are at least 245 nm. In some variations, these wavelengths are at least 248 nm. In some variations, these wavelengths are at least 250 nm. In some variations, these wavelengths are at least 255 nm. In some variations, the wavelengths are less than or equal to 270 nm. In some variations, the wavelengths are less than or equal to 265 nm. In some variations, the wavelengths are less than or equal to 260 nm. In some variations, the wavelengths are 248 nm 268 nm. In some variations, the wavelengths are about 258 nm.

UV radiation has become widely adopted by many hospitals and in high traffic areas (e.g., instance airports and enclosed shopping facilities) as a versatile or extensively understood and efficient method of surface disinfection, complimenting the more common practice of physical cleaning with use of approved chemical cleaning products alone.

Ultraviolet light is electromagnetic radiation with wavelengths shorter than visible light but longer than X-rays. Ultraviolet light is categorized into several wavelength ranges, with short-wavelength ultraviolet (UV-C) considered “germicidal UV.” Wavelengths of about 200 nm to about 300 nm are strongly absorbed by nucleic acids. The absorbed energy can result in defects, including pyrimidine dimers. These dimers can prevent replication or can prevent the expression of necessary proteins, resulting in the death or inactivation of the organism.

The disclosure relates to methods and devices for sanitizing a given volume of air, calculated for the purposes of explanation at 1 m³ per minute of air within a given space. The disclosure refers to “fluid”, which in general means that air can include a certain amount of water vapor (i.e. humidity), but that air as used herein is considered a fluid, as is understood in the art. In variations, the methods and devices can be adapted to different sized devices. In some variations, the methods and devices can be adapted to 100 m³ per minute. In some variations, the methods and devices can be adapted to 1000 m³ per minute. n some variations, the methods and devices can be adapted to 100,000 m³ per minute.

In some variations, with increased volume of air, the residence time of particles can be increased, and not necessarily energy demand. By increasing the time particles are held allows for particle capture, exposing the particle to radiation, and pass the volume of air through the device. Via a tortuous path, the length of time increases. The number of emitters, IR requirement, and bombard through a filter capable can be adjusted for sending a specific volume of air through filter at a specific rate.

The disinfecting apparatuses of the disclosure may also be utilized to maintain a sanitized space, such as by exchanging a given quantity of air changes per hour. As described in greater detail herein, the disinfecting apparatuses may impart multiple doses of electromagnetic energy at specific volumes in mJ/cm calculated by a bacterial, fungal and/or viral sterilization and/or disinfection. The disinfecting apparatuses of the disclosure may, in variations, be described as fixed, multi-frequency UV, UV-C, and/or infrared disinfection apparatus germicidal irradiation. The disinfecting apparatuses of the disclosure may remove all or substantially all traces of biological detritus and/or may reduce organic particles to their basic form, such as by passing a fluid flow over a filtration device (e.g., a metal filter made of copper, zinc, nickel, or combinations thereof) as described herein. In variations, the filtration device may be heated, (in a non-limiting example e.g., to about 220° C.), which may be in the form of additional infrared energy.

The filtration device may be based upon a porous metal design that has a given number of air pockets per cm³. In one variation, multiple layers of woven metal strands can be used, which increase the number of collisions, thereby achieving significant airflow experiencing minimal or no reduction in flow capacity while providing a sufficient surface area (e.g., 200×100×20 mm). In variations, the filtration device may be a metal foam composite filter made from a metal and/or alloy having low oxidation potential (e.g., bronze). As described herein, the alloy(s) of the filtration device may melt without the concurrent melting of other components (e.g., calcium chloride), such that micropore holes may create a geometric foam matrix in the metal filtration device. The filtration device(s) described herein may advantageously be solid-state, self-cleaning (e.g., of carbonized material). The filtration device(s) described herein may further advantageously obviate the need for any HEPA or similar filtration. In some variations, the filtration device can be a heated filter. The filter can be formed of a heat-resistant material, such as a metal. The filter can thus be thermally resistant, having the ability to manage a temperature differential. In variations, the filtration device may be a multi-layer woven metal filter of metal construction. Such a multi-layer filtration device may, in variations, include a minimum of two layers of woven metal material. In other variations, such a multi-layer filtration device may include a minimum of three layers of woven metal material. In other variations, such a multi-layer filtration device may include a minimum of four layers of woven metal material. In other variations, such a multi-layer filtration device may include a minimum of more than four layers of woven metal material. The filtration device may include metal foam and/or metal chips. In variations, the metal foam and/or metal chips may be disposed within a rigid construction (e.g., a case or housing). In variations, the filtration device may define apertures therethrough configured to allow the fluid flow to pass therethrough as the fluid flow is filtered by the filtration device as described herein. Such apertures may, in variations, have a cross-sectional dimension of about 100 to about 2500 microns. As will be appreciated, the size of the apertures may be defined as desired to suit a particular application. By way of non-limiting example, such apertures may have a cross-sectional dimension of about 100 to about 500 microns. In other variations, such apertures may have a cross-sectional dimension of about 700 to about 1000 microns. In other variations, such apertures may have a cross-sectional dimension of about 1000 to about 2500 microns. In surface disinfection applications, UV efficacy can be calculated using a mechanism quantifying the dose in units of watts per second over a given two-dimensional area, μWs/cm²=UV. The intensity of that dose can be calculated in the same arithmetic way, although time may become a critical measure, μW/cm²×Exposure time (second) to be delivered to a given volume of microbial load (population).

The disinfecting apparatuses of the disclosure may employ one or more of (including a combination of) UVGI, infrared, and/or latent infrared (hot metal filter) electromagnetic energy. As described in greater detail herein, the disinfecting apparatuses may expose microbial contamination within a fluid flow to such electromagnetic energy. Exposure of the microbial contamination to, for example, ultraviolet light at germicidal wavelengths has been found to advantageously irradiate the environment and effectively cause microbial deactivation.

UV-irradiation efficacy has a number of challenges, not the least of which is line of sight. If the light wave (energy) cannot gain sufficient direct contact with the surface of a microorganism, it will not inflict sufficient damage to the nucleotides. Therefore, the effectiveness of germicidal irradiation diminishes. Line of sight and sufficient exposure depends on a variety of factors, including length of time a microorganism is exposed to UV radiation, the intensity and wavelength of the UV radiation, the presence of detritus or other particles that can protect the microorganisms from UV, and a particular microorganism's ability to withstand UV radiation during exposure.

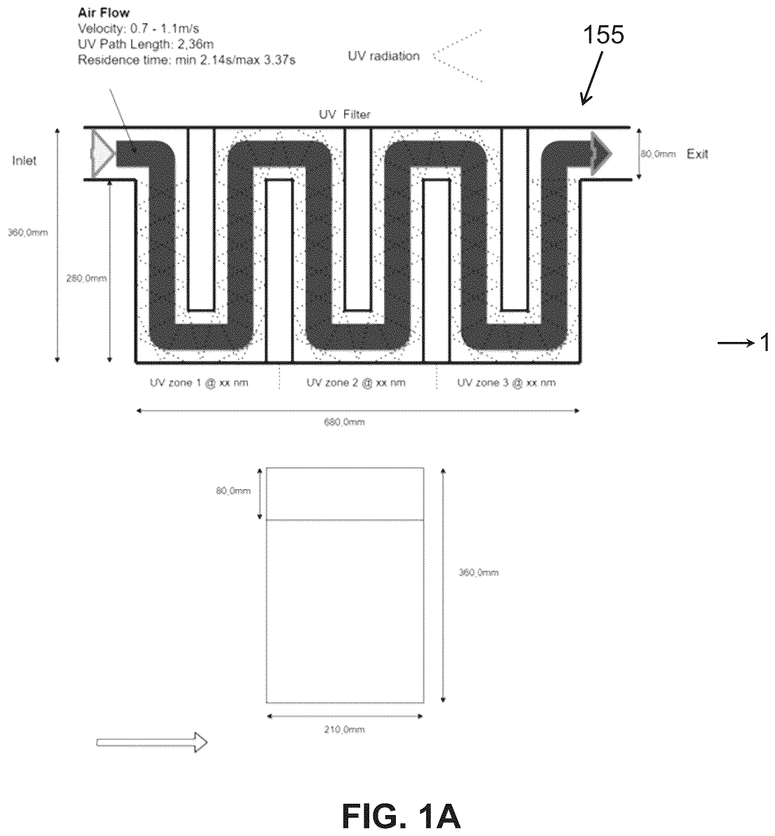

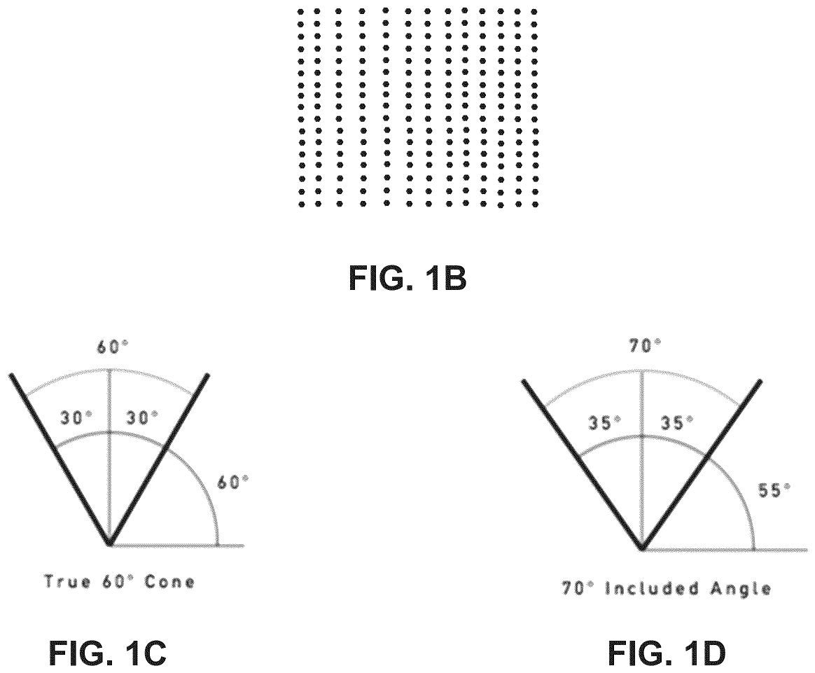

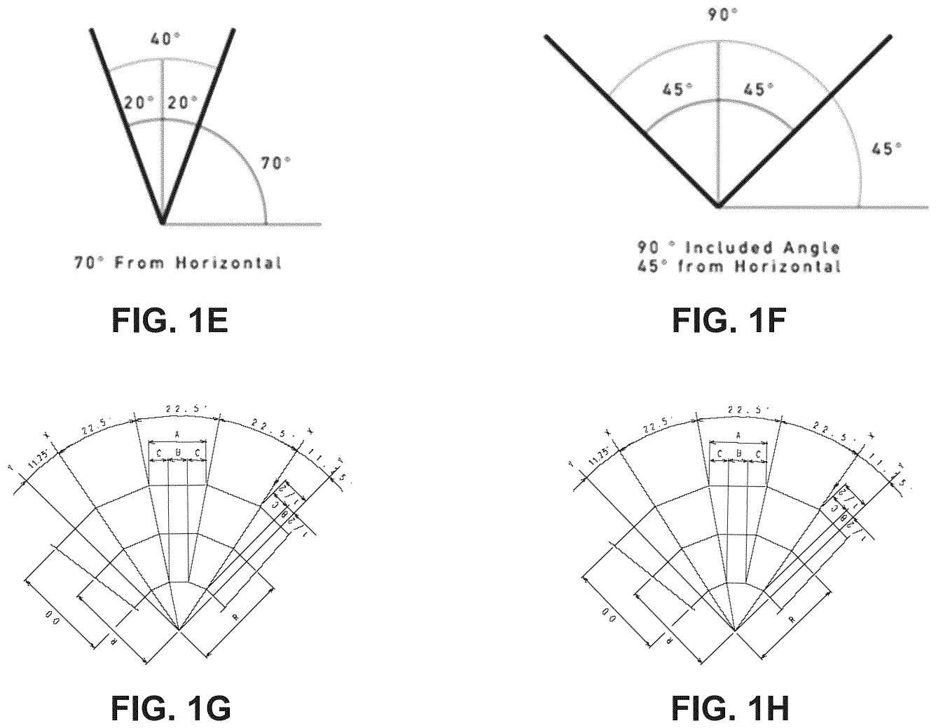

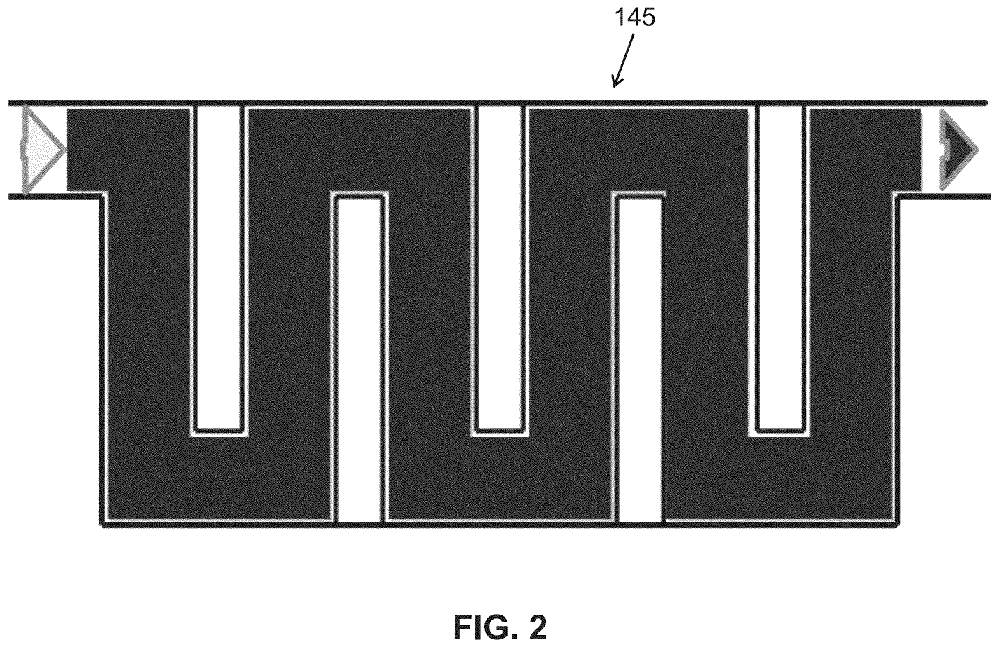

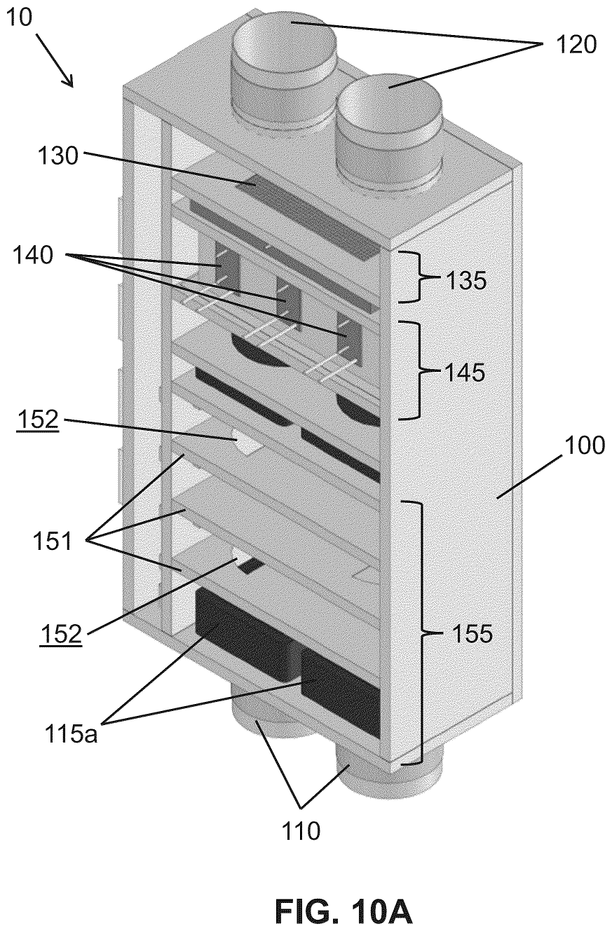

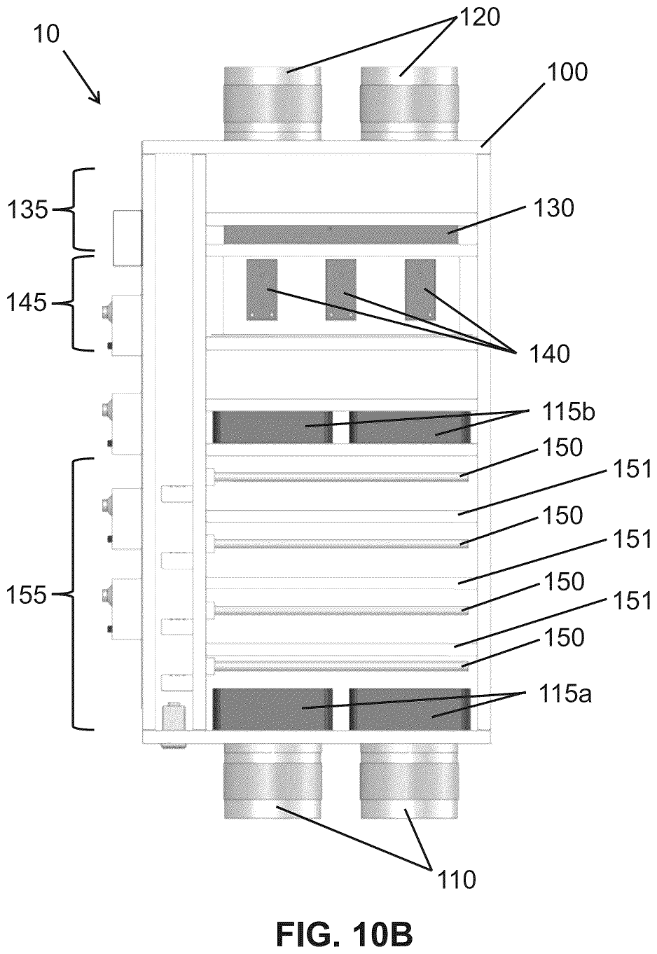

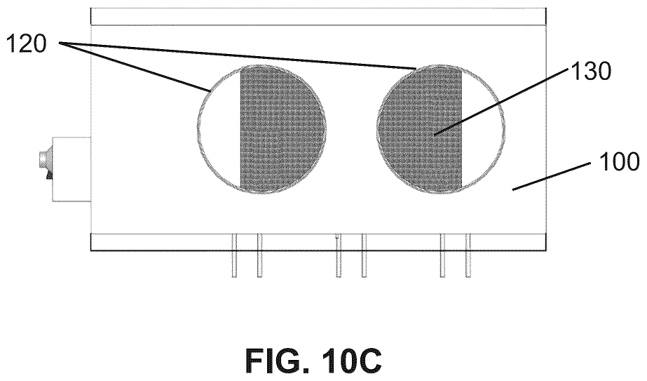

Very generally, to disinfect a fluid flow, the fluid flow (e.g., with microbial contamination) may flow through a body 100 of a disinfection apparatus 10, such as is shown in FIGS. 10A-C. In variations, the body may generally define a non-linear flow path, such as a tortuous flow path through a UV-C stage 155 (refer to FIG. 1A) and/or through an IR stage 145 (refer to FIG. 2). In non-limiting variations, the tortuous flow path may be a substantially serpentine flow path, such as is illustrated, although other variations are not so limited. In variations, the tortuous flow path may delineate multiple turns, such that the fluid flow changes direction as the fluid flow proceeds through the tortuous flow path. As may be appreciated, the tortuous flow path may delineate any desired number of turns to suit a particular application. In a variation, the tortuous flow path delineates two turns. In a variation, the tortuous flow path delineates three turns. In a variation, the tortuous flow path delineates four turns. In a variation, the tortuous flow path delineates five turns (such as is illustrated in FIG. 1A). In a variation, the tortuous flow path delineates six or more turns. As may be appreciated, each turn delineated by the tortuous flow path may generally cause the fluid flow to change direction. For example, as illustrated in FIG. 1A, the fluid flow through the UV-C stage 155 may generally begin by flowing into the UV-C stage 155 (e.g., via an inlet at a first end thereof) along a first direction 1 (i.e., toward an outlet at a second end of the UV-C stage 155 opposite the first end thereof). The fluid flow may then begin to flow along a second direction 2 (i.e., toward a first side of the UV-C stage 155). Upon reaching a first turn, the fluid flow may then change direction to flow around the turn, such as by again flowing in the first direction 1 around the first turn. Upon flowing around the first turn, the flowing flow may then flow along a third direction 3 (i.e., toward a second side of the UV-C stage 155 opposite the first side thereof). As will be understood with continued reference to FIG. 1A, the fluid flow may proceed through the UV-C stage 155 along the tortuous flow path defined thereby until the fluid flow reaches the outlet of the UV-C stage 155. Put another way, the fluid flow may generally proceed through the UV-C stage 155 from the inlet to the outlet thereof. As the fluid flow proceeds through the UV-C stage 155 along the tortuous flow path defined thereby, the fluid flow may flow past as many turns (and change directions as many times) as desired to suit a particular application. In this way, the previously-described problems arising from poor line of sight UV irradiation may be overcome. It has been found that the use of at least two turns (such that the fluid flow changes direction at least twice as the fluid flow proceeds through the tortuous flow path defined by the UV-C stage 155) generally ensures that the fluid flow (including microbial contamination therein) undergoes sufficient turbulence, such as by “tumbling” through at least 360°, thereby ensuring total line of sight for the UV and/or IR radiation. It has been experimentally discovered that flowing the fluid flow through a tortuous flow path as electromagnetic energy is imparted upon a fluid flow fluid flow as described herein may enhance the effectiveness of exposure to UV and/or IR radiation on particulates (e.g., microorganisms) of the fluid flow as the fluid flow proceeds through the tortuous flow path, such as by increasing the length of the path and/or increasing residence or exposure time.

As may be appreciated, the tortuous flow path of the UV-C stage 155 may be defined by a plurality of spaced-apart walls 151, such as is illustrated in FIG. 10A and FIG. 10B. In some variations (refer to FIG. 1A), each wall defining the tortuous flow path may span less than a full dimension of the UV-C stage 155 (i.e., across the body 100 of the disinfection apparatus 10) such that the flow path proceeds around ends of each of the walls as the flow path proceeds from the inlet to the outlet of the UV-C stage 155. In other variations (refer to FIG. 10A and FIG. 10B), each wall defining the tortuous flow path may span a full dimension of the UV-C stage 155 (i.e., across the body 100 of the disinfection apparatus 10) and may define an aperture 152 configured to permit the flow path to extend therethrough. In variations, the apertures 152 may be semicircular and designed so as to create or increase turbulence and/or pressure within the UV-C stage.

As may be appreciated, the body 100 of the disinfection apparatus 10 may be of any size, shape, number, and/or type to suit a particular application. By way of non-limiting example, the body 100 may be constructed of a metal, such as an antibacterial metal (e.g., copper, brass, bronze, silver, and/or combinations thereof). In some instances, the metal is an antimicrobial metal. In variations, the interior surfaces of the body 100 may be coated with an antimicrobial or antipathogenic coating, such as an AgCl coated TiO powder coat.

Additionally, the disinfection apparatus 10 (including but not limited to the body 100 thereof) may be constructed of a non-porous material that discourages agglomeration of detritus. In some variations, the disinfection apparatus 10 is capable of relecting electromagnetic radiation. For example, the material can be a metal that would be complementary to the reflection of electromagnetic wavelengths and combinations thereof preferably polished.

In variations, the disinfection apparatus may include one or more motors (e.g., forced air motors) configured to force the fluid flow into and/or through the disinfection apparatus. The velocity and/or intensity of the fluid flow may, in variations, be controlled via a monitoring system that may, from time to time, reduce the exposure time by varying the velocity and/or intensity of the fluid flow and/or change the wavelength of the UV-C and/or IR administered, thereby minimizing power consumption and improving environmental conditions. At or near the inlet(s) 110 of the disinfection apparatus 10, one or more inlet fans 115a may be positioned and configured to generate a desired air flow, such as is illustrated in FIG. 10A. As may be appreciated, the one or more inlet fans 115a may be configured to generate any desired flow to suit a particular application.

The inlet fans 115a can be designed force gas through a specific average distance per unit time. By way of non-limiting example, the one or more inlet fans 115a may be positioned and configured to generate a desired air flow. In some variations, the inlet air flow is at least 0.3 m/s. In some variations, the inlet air flow is at least 0.4 m/s. In some variations, the inlet air flow is at least about 0.6 m/s. In some variations, the inlet air flow is at least about 0.8 m/s. In some variations, the inlet air flow is at least about 1.0 m/s. In some variations, the inlet air flow is at least about 2.0 m/s. In some variations, the inlet air flow is at least about 3.0 m/s. In some variations, the inlet air flow is at least about 4.0 m/s. In some variations, the inlet air flow is at least about 5.0 m/s. In some variations, the inlet air flow is less than or equal to 6.0 m/s. In some variations, the inlet air flow is less than or equal to 5.0 m/s. In some variations, the inlet air flow is less than or equal to 4.0 m/s. In some variations, the inlet air flow is less than or equal to 3.0 m/s. In some variations, the inlet air flow is less than or equal to 2.0 m/s. In some variations, the inlet air flow is less than or equal to 1.0 m/s. In some variations, the inlet air flow is less than or equal to 0.8 m/s. In some variations, the inlet air flow is less than or equal to 0.6 m/s. In some variations, the inlet air flow is less than or equal to 0.4 m/s. In further variations, the range can include a lower and upper boundary selected from the above, such as about 0.3 m/s to about 0.6 m/s, about 0.6 m/s to about 2.0 m/s, or 0.6 m/s to about 6 m/s.

In some variations, the inlet air flow can be in mass per minute, such as kg/min. In some variations, the air inlet air flow is at least 0.2 kg/min. In some variations, the air inlet air flow is at least 0.4 kg/min. In some variations, the air inlet air flow is at least 0.6 kg/min. In some variations, the air inlet air flow is at least 0.8 kg/min. In some variations, the air inlet air flow is at least 1.0 kg/min. In some variations, the air inlet air flow is at least 2.0 kg/min. In some variations, the air inlet air flow is at least 4.0 kg/min. In some variations, the air inlet air flow is at least 6.0 kg/min. In some variations, the air inlet air flow is at least 8.0 kg/min. In some variations, the air inlet air flow is at least 10.0 kg/min. In some variations, the air inlet air flow is at least 12.0 kg/min. In some variations, the air inlet air flow is at least 14.0 kg/min. In some variations, the air inlet air flow is at least 16.0 kg/min. In some variations, the air inlet air flow is at least 18.0 kg/min.

In some variations, the inlet air flow is less than or equal to 20.0 kg/min. In some variations, the inlet air flow is less than or equal to 18.0 kg/min. In some variations, the inlet air flow is less than or equal to 16.0 kg/min. In some variations, the inlet air flow is less than or equal to 14.0 kg/min. In some variations, the inlet air flow is less than or equal to 12.0 kg/min. In some variations, the inlet air flow is less than or equal to 10.0 kg/min. In some variations, the inlet air flow is less than or equal to 8.0 kg/min. In some variations, the inlet air flow is less than or equal to 6.0 kg/min. In some variations, the inlet air flow is less than or equal to 4.0 kg/min. In some variations, the inlet air flow is less than or equal to 2.0 kg/min. In some variations, the inlet air flow is less than or equal to 1.0 kg/min. In some variations, the inlet air flow is less than or equal to 0.8 kg/min. In some variations, the inlet air flow is less than or equal to 0.6 kg/min. In some variations, the inlet air flow is less than or equal to 0.4 kg/min.

In further variations, the range can include a lower and upper boundary selected from the above, such as about 0.2 kg/min to about 0.8 kg/min, 0.8 kg/min to about 2.0 kg/min. or about 0.2 kg/min to about 20 kg/min

In some variations, the inlet air flow is about 300 L/min to about 700 L/min but that could be additionally 300 L/min to about 3000 L/min or about 3,000,000 L/min.

In another variation, the one or more inlet fans 115a may be positioned and configured to generate a desired air flow of described above. For example, the air flow can be about about 700 L/min to about 1200 L/min. In yet another variation, the one or more inlet fans may be positioned and configured to generate a desired air flow of about 2.0 m/s to about 4.0 m/s, about 2.0 kg/min to about 4.0 kg/min, and/or about 1200 L/min to about 10000 L/min. In a further iteration, the inlet fans can be 1600 m³/h to about 100,000 m³/h. The inlet fan(s) 115a may, in variations, draw the fluid flow (e.g., air from the environment) into the body 100 of the disinfection apparatus 10. The fan(s) and/or the motor(s) may be operated to manage the exposure time of the fluid flow, such as by ensuring that the fluid flow flows through the body 100 of the disinfection apparatus 10 at a flow rate of less than or equal to about 0.3 m/s. In a further iteration a flow rate could be less than or equal to about 30 m/s. In variations, the size and/or shape of treatment passages defining the tortuous flow path through the body 100 of the disinfection apparatus 10 may further or alternatively manage the exposure time and effectiveness of the fluid flow, thereby ensuring a desirable level of energy transfer into the fluid flow, such as into microbial foreign objects (MFO) contained within the fluid flow. In variations, the exposure may be about 3 mj/cm seconds or more.

As the fluid flow (including microbial contamination therein) proceeds through the body 100 of the disinfection apparatus 10, the fluid flow may be exposed to electromagnetic energy imparted into the flow path. In variations, ultraviolet light and/or infrared light may be emitted upon the fluid flow as the fluid flow proceeds through the tortuous flow path. As may be appreciated, in some variations, the disinfection apparatus 10 may include a single body 100 (such as those illustrated in FIG. 1A, 2, 5A-5D, or 6) through which the fluid flow may flow for exposure to multiple “stages” of electromagnetic energy within the single body. In other variations, the disinfection apparatus 10 may include multiple bodies 100 (such as those illustrated in FIG. 1A, 2, 5A-5D, or 6) through which the fluid flow may flow (e.g., in succession) for exposure to one or more “stages” of electromagnetic energy within each body. The stages of exposure may, in variations, be thermally and/or physically separated from one another, such as by thermal barrier gaskets.

### UV-C Stage

In one “stage” of exposure of the fluid flow to electromagnetic energy (referred to herein as the UV-C stage 155), one or more emitters 150 may be arranged to emit electromagnetic energy upon the fluid flow. Generally, the electromagnetic energy is emitted upon the fluid flow as the fluid flow proceeds along the tortuous flow path defined by the body 100 of the disinfection apparatus 10. The emitter(s) 150 may, in variations, be configured to emit ultraviolet light (e.g., within a germicidal and/or disinfecting wavelength) upon the fluid flow. The emitter(s) may emit short-wavelength ultraviolet (UV-C) light, also considered “germicidal UV” light. In this regard, the stage including one or more emitters configured to emit ultraviolet light (e.g., UV-C light, such as within a germicidal and/or disinfecting wavelength) may be considered a UV-C stage. As may be appreciated, the emitter(s) 150 of the UV-C stage 155 may be configured to emit light (e.g., UV light) at any one or more desired wavelengths to suit a particular application. Selective species are sensitive to one or more specific wavelengths. For example, E. coli has sensitivity to UV LEDs at about 265 nm, whereas virus tincture is more sensitive to about 222 nm to about 230 nm and about 248 nm to about 254 nm. The way microorganisms absorb UV energy is similar in process but can be more damaging to how sunburn can affect skin, although this is via wavelengths of longer lengths and within the light received from the Sun (UV-B).

In a variation, the one or more emitters 150 of the UV-C stage 155 are configured to emit ultraviolet light at one or more wavelengths of at least 120 nm. In a variation, the one or more emitters of the UV-C stage are configured to emit ultraviolet light at one or more wavelengths of at least 130 nm. In a variation, the one or more emitters of the UV-C stage are configured to emit ultraviolet light at one or more wavelengths of at least 140 nm. In a variation, the one or more emitters of the UV-C stage are configured to emit ultraviolet light at one or more wavelengths of at least 150 nm. In a variation, the one or more emitters of the UV-C stage are configured to emit ultraviolet light at one or more wavelengths of at least 160 nm. In a variation, the one or more emitters of the UV-C stage are configured to emit ultraviolet light at one or more wavelengths of at least 170 nm. In a variation, the one or more emitters of the UV-C stage are configured to emit ultraviolet light at one or more wavelengths of at least 180 nm. In a variation, the one or more emitters of the UV-C stage are configured to emit ultraviolet light at one or more wavelengths of at least 190 nm. In a variation, the one or more emitters of the UV-C stage are configured to emit ultraviolet light at one or more wavelengths of at least 200 nm. In a variation, the one or more emitters of the UV-C stage are configured to emit ultraviolet light at one or more wavelengths of at least 210 nm. In a variation, the one or more emitters of the UV-C stage are configured to emit ultraviolet light at one or more wavelengths of at least 220 nm. In a variation, the one or more emitters of the UV-C stage are configured to emit ultraviolet light at one or more wavelengths of at least 230 nm. In a variation, the one or more emitters of the UV-C stage are configured to emit ultraviolet light at one or more wavelengths of at least 240 nm. In a variation, the one or more emitters of the UV-C stage are configured to emit ultraviolet light at one or more wavelengths of at least 250 nm. In a variation, the one or more emitters of the UV-C stage are configured to emit ultraviolet light at one or more wavelengths of at least 260 nm. In a variation, the one or more emitters of the UV-C stage are configured to emit ultraviolet light at one or more wavelengths of at least 270 nm.

In a variation, the one or more emitters 150 of the UV-C stage 155 are configured to emit ultraviolet light at one or more wavelengths of equal to or less than 280 nm. In a variation, the one or more emitters of the UV-C stage are configured to emit ultraviolet light at one or more wavelengths of equal to or less than 270 nm. In a variation, the one or more emitters of the UV-C stage are configured to emit ultraviolet light at one or more wavelengths of equal to or less than 260 nm. In a variation, the one or more emitters of the UV-C stage are configured to emit ultraviolet light at one or more wavelengths of equal to or less than 250 nm. In a variation, the one or more emitters of the UV-C stage are configured to emit ultraviolet light at one or more wavelengths of equal to or less than 240 nm. In a variation, the one or more emitters of the UV-C stage are configured to emit ultraviolet light at one or more wavelengths of equal to or less than 230 nm. In a variation, the one or more emitters of the UV-C stage are configured to emit ultraviolet light at one or more wavelengths of equal to or less than 220 nm. In a variation, the one or more emitters of the UV-C stage are configured to emit ultraviolet light at one or more wavelengths of equal to or less than 210 nm. In a variation, the one or more emitters of the UV-C stage are configured to emit ultraviolet light at one or more wavelengths of equal to or less than 200 nm. In a variation, the one or more emitters of the UV-C stage are configured to emit ultraviolet light at one or more wavelengths of equal to or less than 190 nm. In a variation, the one or more emitters of the UV-C stage are configured to emit ultraviolet light at one or more wavelengths of equal to or less than 180 nm. In a variation, the one or more emitters of the UV-C stage are configured to emit ultraviolet light at one or more wavelengths of equal to or less than 170 nm. In a variation, the one or more emitters of the UV-C stage are configured to emit ultraviolet light at one or more wavelengths of equal to or less than 160 nm. In a variation, the one or more emitters of the UV-C stage are configured to emit ultraviolet light at one or more wavelengths of equal to or less than 150 nm. In a variation, the one or more emitters of the UV-C stage are configured to emit ultraviolet light at one or more wavelengths of equal to or less than 140 nm. In a non-limiting example, for a given flow rate of 0.7 m/s per kg air, it was found that a desirable UV inoculation would be 0.3 W/cm² generated at a wavelength of 222 nm. In another non-limiting example, for a given flow rate of 0.6 m/s per kg air, it was found that a desirable UV inoculation would be 0.3 mJ/cm² generated at a wavelength of 254 nm.

In variations, the emitter(s) 150 of the UV-C stage 155 may be configured to emit UV light having one or more peaks. By way of non-limiting example, it has been experimentally discovered that emitting UV light having a first wavelength peak at about 222 nm, a second wavelength peak at about 254 nm, and a third wavelength peak at about 265 nm demonstrated strong disinfecting effects.

As may be appreciated, the emitter(s) 150 of the UV-C stage 155 may be of any size, shape, number, and/or type to suit a particular application. By way of non-limiting example, the emitter(s) of the UV-C stage may include one or more of mercury-based lamps (e.g., operating at low vapor pressure), ultraviolet light emitting diode lamps, pulsed-xenon lamps, and/or combinations thereof. In variations employing mercury-based lamps, the emitted light can be at a wavelength of about 253.7 nm. In variations employing UV LEDs, the emitted light can be at one or more wavelengths of about 255 nm to about 280 nm. In variations employing pulsed-xenon lamps, the emitted light may generally be across the entire UV spectrum, including with a peak emission of about 230 nm. Certain UV LEDs may use semiconductors to emit light between 255 nm to 280 nm, which may not be efficient at a 225 nm excitation point of 222 nm. Thus, where it is desirable to achieve the 225 nm excitation point, an alternative UV light generator may be utilized, such as an excimer lamp. Excimer lamps may, in variations, also provide the further advantages of generating minimal ozone and posing no or minimal risk to mammalian cells. According to one non-limiting example, it was hypothesized that a desired exposure would be a series of UV irradiation emitters administering total dose rates per particle of 0.3 ws/cm². In such a non-limiting variation, it was discovered that a total of six banks of emitters spaced about 40 mm apart demonstrated strong disinfecting effects. In one variation, the emitters of the UV-C stage may include a plurality of ultraviolet light emitting diodes (e.g., about 500 to about 5000 LED emitters) and an excimer lamp. The plurality of ultraviolet light emitting diodes may, in variations, be arranged in an array. The array could, in variations, have a uniform distribution of the ultraviolet light emitting diodes or any other distribution, such as is illustrated in FIG. 1B. In variations, one or more arrays of ultraviolet light emitting diodes may be employed and, in such variations, the arrays may be offset and/or opposite one another to maximize the cone angle of the emitted light. The cone angle of the emitted light may generally be of any desired value to suit a particular application, as may be understood with reference to FIGS. 1C-1H. By way of non-limiting example, the cone angle of the emitted light may be about 40° to about 90°. By way of further non-limiting example, the cone angle of the emitted light may have a convergence at between about 40 mm and 80 mm.

The wavelength emission may be tunable, such as by adjusting the material of the emitter (e.g., the material of the diode and/or semiconductor). The use of LEDs which emit a wavelength more precisely tuned to the maximal germicidal wavelength may result in greater microbe deactivation per amp of power, maximization of microbial deactivation, and/or less ozone production.

In variations, the UV-C stage 155 may further include one or more emitters configured to emit visible light upon the fluid flow. Such emitter(s) may be configured to emit visible light at one or more wavelengths of about 380 nm to about 405 nm. Emitting visible light within such a wavelength range may expose the fluid flow to antimicrobial properties (e.g., for environmental disinfection and infection control), such as by through a light-induced photodynamic inactivation process involved in the destruction of a wide range of prokaryotic and eukaryotic microbial species, including resistant forms commonly identified as MRSA and VRSA (e.g., bacterial and Mycobacteria and Stacbotrys and aspergillus fungal spores). In certain variations, blue light (e.g., at a wavelength of from 380 nm to 405 nm) may be utilized to provide additional significant antimicrobial properties against a wide range of bacterial and fungal pathogens. It has been found that while the germicidal efficacy of visible light may be lower than for UV light, this limitation may be offset by visible light's facility for safe, continuous use in occupied environments. In one variation, the emitters of the UV-C stage configured to emit visible light may include a plurality of visible light emitting diodes (e.g., about 100 to about 500 LED emitters). The plurality of visible light emitting diodes may, in variations, be arranged in an array. The array could, in variations, have a uniform distribution of the visible light emitting diodes or any other distribution.

In some variations, a non-LED emitter can be used. The wavelength range may be any wavelength range described here, for example, 230 nm up to 278 nm. In some variations, the intensity is at least the equivalence of 0.3 mJ/cm³/s. In some variations, the intensity is at least the equivalence of 0.4 mJ/cm³/s. In some variations, the intensity is at least the equivalence of 0.5 mJ/cm³/s. In some variations, the intensity is at least the equivalence of 0.4 mJ/cm³/s to 5 mJ/cm³/s.

### IR Stage

In another “stage” of exposure of the fluid flow to electromagnetic energy (referred to herein as the IR stage 145), one or more emitters 140 may be arranged to emit electromagnetic energy upon the fluid flow. As previously described, this stage of exposure may occur within the same or a separate body 100 of the disinfection apparatus 10 as any one or more of the other stages. In variations, this stage of exposure (also referred to herein as the IR stage) may occur before or after the UV-C stage of exposure (also referred to herein as the UV-C stage 155). Generally, in the IR stage 145, the electromagnetic energy is emitted upon the fluid flow as the fluid flow proceeds along a tortuous flow path defined by the body 100 of the disinfection apparatus 10 (such as is illustrated in FIG. 2). The emitter(s) of the IR stage may, in variations, be configured to emit infrared light (e.g., within a germicidal and/or sterilizing wavelength) upon the fluid flow. As may be appreciated, the emitter(s) of the IR stage may be configured to emit light (e.g., IR light) at any one or more desired wavelengths to suit a particular application. The emitter(s) of the IR stage may be configured to emit infrared light effective to boil a lipid bilayer, protein envelope, and/or peptidoglycan cell walls (of gram-negative and gram-positive bacteria) present in one or more microorganisms of the fluid flow. In the IR stage, the fluid flow (e.g., microbial foreign objects contained within the fluid flow) may be desiccated, thereby sterilizing the fluid flow.

In a variation, the one or more emitters 140 of the IR stage 145 are configured to emit infrared light at one or more wavelengths of at least 1100 nm. In a variation, the one or more emitters of the IR stage are configured to emit infrared light at one or more wavelengths of at least 1150 nm. In a variation, the one or more emitters of the IR stage are configured to emit infrared light at one or more wavelengths of at least 1200 nm. In a variation, the one or more emitters of the IR stage are configured to emit infrared light at one or more wavelengths of at least 1250 nm. In a variation, the one or more emitters of the IR stage are configured to emit infrared light at one or more wavelengths of at least 1300 nm. In a variation, the one or more emitters of the IR stage are configured to emit infrared light at one or more wavelengths of at least 1350 nm. In a variation, the one or more emitters of the IR stage are configured to emit infrared light at one or more wavelengths of at least 1400 nm. In a variation, the one or more emitters of the IR stage are configured to emit infrared light at one or more wavelengths of at least 1410 nm. In a variation, the one or more emitters of the IR stage are configured to emit infrared light at one or more wavelengths of at least 1420 nm. In a variation, the one or more emitters of the IR stage are configured to emit infrared light at one or more wavelengths of at least 1430 nm. In a variation, the one or more emitters of the IR stage are configured to emit infrared light at one or more wavelengths of at least 1440 nm. In a variation, the one or more emitters of the IR stage are configured to emit infrared light at one or more wavelengths of at least 1450 nm. In a variation, the one or more emitters of the IR stage are configured to emit infrared light at one or more wavelengths of at least 1460 nm. In a variation, the one or more emitters of the IR stage are configured to emit infrared light at one or more wavelengths of at least 1470 nm. In a variation, the one or more emitters of the IR stage are configured to emit infrared light at one or more wavelengths of at least 1480 nm. In a variation, the one or more emitters of the IR stage are configured to emit infrared light at one or more wavelengths of at least 1490 nm.

In a variation, the one or more emitters 140 of the IR stage 145 are configured to emit infrared light at one or more wavelengths of less than or equal to 1600 nm. In a variation, the one or more emitters of the IR stage are configured to emit infrared light at one or more wavelengths of less than or equal to 1590 nm. In a variation, the one or more emitters of the IR stage are configured to emit infrared light at one or more wavelengths of less than or equal to 1580 nm. In a variation, the one or more emitters of the IR stage are configured to emit infrared light at one or more wavelengths of less than or equal to 1570 nm. In a variation, the one or more emitters of the IR stage are configured to emit infrared light at one or more wavelengths of less than or equal to 1560 nm. In a variation, the one or more emitters of the IR stage are configured to emit infrared light at one or more wavelengths of less than or equal to 1550 nm. In a variation, the one or more emitters of the IR stage are configured to emit infrared light at one or more wavelengths of less than or equal to 1540 nm. In a variation, the one or more emitters of the IR stage are configured to emit infrared light at one or more wavelengths of less than or equal to 1530 nm. In a variation, the one or more emitters of the IR stage are configured to emit infrared light at one or more wavelengths of less than or equal to 1520 nm. In a variation, the one or more emitters of the IR stage are configured to emit infrared light at one or more wavelengths of less than or equal to 1510 nm. In a variation, the one or more emitters of the IR stage are configured to emit infrared light at one or more wavelengths of less than or equal to 1500 nm. In a variation, the one or more emitters of the IR stage are configured to emit infrared light at one or more wavelengths of less than or equal to 1490 nm. In a variation, the one or more emitters of the IR stage are configured to emit infrared light at one or more wavelengths of less than or equal to 1480 nm. In a variation, the one or more emitters of the IR stage are configured to emit infrared light at one or more wavelengths of less than or equal to 1470 nm. In a variation, the one or more emitters of the IR stage are configured to emit infrared light at one or more wavelengths of less than or equal to 1460 nm. In a variation, the one or more emitters of the IR stage are configured to emit infrared light at one or more wavelengths of less than or equal to 1450 nm. In a variation, the one or more emitters of the IR stage are configured to emit infrared light at one or more wavelengths of less than or equal to 1440 nm. In a variation, the one or more emitters of the IR stage are configured to emit infrared light at one or more wavelengths of less than or equal to 1430 nm. In a variation, the one or more emitters of the IR stage are configured to emit infrared light at one or more wavelengths of less than or equal to 1420 nm. In a variation, the one or more emitters of the IR stage are configured to emit infrared light at one or more wavelengths of less than or equal to 1410 nm. In a variation, the one or more emitters of the IR stage are configured to emit infrared light at one or more wavelengths of less than or equal to 1400 nm. In a variation, the one or more emitters of the IR stage are configured to emit infrared light at one or more wavelengths of less than or equal to 1350 nm. In a variation, the one or more emitters of the IR stage are configured to emit infrared light at one or more wavelengths of less than or equal to 1300 nm. In a variation, the one or more emitters of the IR stage are configured to emit infrared light at one or more wavelengths of less than or equal to 1250 nm.

The one or more emitters 140 of the IR stage 145 can have any of the upper boundary and any of the lower boundary. Further, multiple emitters can each have a different upper boundary and lower boundary. One or more emitters can have the same upper boundary and lower boundary—for example, in a three emitter variation, two emitters can have the same range, and the third emitter can have a different range. By way of another example, one emitter can be configured to emit infrared light having a first wavelength from 1200 nm to 1300 nm (or as an alternative, peak at about 1250 nm), and a second wavelength from 1300 nm to 1400 nm (or as an alternative, with a peak of 1354 nm. In some variations, the one or more emitters are configured to emit infrared light at a first wavelength of the at least one wavelength of the second stage is from 1200 nm to 1510 nm upon the fluid flow. In some variations, the one or more emitters are configured to emit infrared light at a second wavelength of the at least one wavelength of the second stage is from 1380 nm to 1800 nm upon the fluid flow.

In various aspects, the relative humidity of the air at room temperature and pressure is reduced by between 5% and 50%, in some cases 10% and 35%. Experimental results indicated prominent spectral differences between dry lipid and hydroxol group, including H₂O. Principal components analysis and hierarchical clustering indicated that infrared wavelength between 1200 nm and 1800 nm were influential, including bands of infrared light at wavelengths of 1200 nm, 1450 nm, 1650 nm, 1695 nm, 1700 nm, and/or 1800 nm. Bands in the fingerprint region (800-1800 nm) were correctly assigned. Calculations revealed the existence of two coupled vibration between the absorption characteristics of hydroxyl group lipid and methylene (1120 and 1160 nm). The relative humidity results in substantial reduction in microbial viability and microbial count, including mold, bacteria, and viruses.

The one or more emitters 140 of the IR stage 145 can be any IR emitter known in the art. For example, the one or more emitters can be infrared light emitting diodes. The one or more emitters may include at least one halogen lamp.

In further variations, the stage may further include one or more emitters configured to emit visible light at one or more wavelengths of about 380 nm to about 405 nm. In a non-limiting example, it was found that delivering IR radiation at 1300 nm to 1600 nm (e.g., at 1410 nm, 1540 nm, or 1595 nm) exhibited advantageous H₂O absorption. An increase in temperature of H₂O molecules and corresponding lipids (e.g., phosphorylated N-acetyl glucosamine compounds) from 23° C. to in excess of 54° C. over a period of 0.6 to 1.2 seconds, which was found to inflict catastrophic damage to the cell and the cell structure. Advantageous absorption for hydroxyl groups (H₂O) were experimentally discovered to be at IR radiation of 1410 to 1420 nm. Advantageous absorption for lipid groups and supporting organic material in the protein and lipid cellular material were experimentally discovered to be at IR radiation of 1480 to 1540 nm.

In variations, the one or more emitters may be configured to emit infrared light having a first wavelength in a range of 1200 nm-1300 nm (e.g., peak at about 1250 nm), a second wavelength in a range from 1300 nm-1400 nm (e.g., peak at about 1354 nm, and a third wavelength peak in a range from 1400 nm-1500 nm (e.g., peak at about 1465 nm). The one or more emitters may include a plurality of infrared light emitting diodes. In the same or other variations, the one or more emitters may include at least one halogen lamp. In an aspect, the stage may further include one or more emitters configured to emit visible light at one or more wavelengths of about 380 nm to about 405 nm.

As may be appreciated, the emitter(s) of the IR stage 145 may be of any size, shape, number, and/or type to suit a particular application. By way of non-limiting example, the amount of energy discharged by the emitter(s) of the IR stage may be about 0.1 W/scm² to about 7.0 w/scm². In a variation, the amount of energy discharged by the emitter(s) of the IR stage may be about 0.5 W/scm² to about 6.5 w/scm². In a variation, the amount of energy discharged by the emitter(s) of the IR stage may be about 1.0 W/scm² to about 6.0 w/scm². In a variation, the amount of energy discharged by the emitter(s) of the IR stage may be about 1.5 W/scm² to about 5.5 w/scm². In a variation, the amount of energy discharged by the emitter(s) of the IR stage may be about 2.0 W/scm² to about 5.0 w/scm². In a variation, the amount of energy discharged by the emitter(s) of the IR stage may be about 2.5 W/scm² to about 4.5 w/scm². In a variation, the amount of energy discharged by the emitter(s) of the IR stage may be about 3.0 W/scm² to about 4.0 w/scm². In a non-limiting example, it was experimentally discovered that discharging, by the emitter(s) of the IR stage, an amount of energy of about 0.5 w/scm² to about 2 w/scm² exhibited desirable effects. In one variation, the emitters of the UV-C stage may include a plurality of infrared light emitting diodes (e.g., about 600 to about 2000 LED emitters). The plurality of infrared light emitting diodes may, in variations, be arranged in an array. The array could, in variations, have a uniform distribution of the infrared light emitting diodes or any other distribution. In a variation, an emitter may be tunable to deliver a particular amount of radiation.

In some variations, the amount of energy discharged by an emitter is at least 0.1 W/s/cm³. In some variations, the amount of energy discharged by an emitter is at least 0.5 W/s/cm³. In some variations, the amount of energy discharged by an emitter is at least 1.0 W/s/cm³. In some variations, the amount of energy discharged by an emitter is at least 2.0 W/s/cm³. In some variations, the amount of energy discharged by an emitter is at least 3.0 W/s/cm³. In some variations, the amount of energy discharged by an emitter is at least 4.0 W/s/cm³. In some variations, the amount of energy discharged by an emitter is at least 5.0 W/s/cm³. In some variations, the amount of energy discharged by an emitter is at least 6.0 W/s/cm³. In some variations, the amount of energy discharged by an emitter is less than or equal to 7 W/s/cm³. In some variations, the amount of energy discharged by an emitter is less than or equal to 6 W/s/cm³. In some variations, the amount of energy discharged by an emitter is less than or equal to 5 W/s/cm³. In some variations, the amount of energy discharged by an emitter is less than or equal to 4 W/s/cm³. In some variations, the amount of energy discharged by an emitter is less than or equal to 3 W/s/cm³. In some variations, the amount of energy discharged by an emitter is less than or equal to 2 W/s/cm³. In some variations, the amount of energy discharged by an emitter is less than or equal to 1 W/s/cm³. In some variations, the amount of energy discharged by an emitter is less than or equal to 0.5 W/s/cm³.

It will be recognized that a particular emitter can have a different energy, or energy range, than other emitters. Further, it will be recognized that a lower and upper boundary described above can be selected for each emitter. By way of non-limiting example, the amount of energy discharged by the emitter(s) of the IR stage may be about 0.1 W/s/cm³ to about 7.0 W/s/cm³. In a variation, the amount of energy discharged by the emitter(s) of the IR stage may be about 0.5 W/s/cm³ to about 6.5 W/s/cm³. In a variation, the amount of energy discharged by the emitter(s) of the IR stage may be about 1.0 W/s/cm³ to about 6.0 W/s/cm³. In a variation, the amount of energy discharged by the emitter(s) of the IR stage may be about 1.5 W/s/cm³ to about 5.5 W/s/cm³. In a variation, the amount of energy discharged by the emitter(s) of the IR stage may be about 2.0 W/s/cm³ to about 5.0 W/s/cm³. In a variation, the amount of energy discharged by the emitter(s) of the IR stage may be about 2.5 W/s/cm³ to about 4.5 W/s/cm³.

The wavelength emission may be tunable, such as by adjusting the material of the emitter (e.g., the material of the diode and/or semiconductor). The use of LEDs which emit a wavelength more precisely tuned to the maximal germicidal wavelength may result in greater microbe deactivation per amp of power, maximization of microbial deactivation, and/or less ozone production.

Resulting epidermal temperature is higher when the surface structure of a cellular organism is irradiated with far infrared irradiation (FIR) than if similar thermal loads from shorter wavelengths are used. Additionally, the wavelength ranges described herein may promote local reactive oxygen species (ROS) generation with a relatively small increase in temperature over a period of 0.3 ms. The prolonged erythemal response due to FIR exposure may be due to increased epidermal temperatures associated therewith, but levels of FIR that do not produce any detectable skin heating can also have significant biological effects.

Infrared absorption, water molecules, and lipid bilayers have three fundamental molecular vibrations. The O—H stretching vibrations give rise to absorption bands with band origins at 3657 cm⁻¹ (v1, 2.734 μm) and 3756 cm⁻¹ (v3, 2.662 μm) in the gas phase. The asymmetric stretching vibration, of B2 symmetry in the point group C2v is a normal vibration. The H—O—H bending mode origin is at 1595 cm⁻¹ (v2, 6.269 μm). Both symmetric stretching and bending vibrations have A1 symmetry, but the frequency difference between them is so large that mixing is effectively zero. In the gas phase, all three bands show extensive rotational fine structure.

In the near-infrared spectrum, the presence of water vapor in the lipid and aqueous phase is important for absorption chemistry especially as the far infrared and near infrared spectra are easy to observe. IR Radiation and its interaction in oxide chemistry exhibits complex IR absorption bands located at 7.9 μm that overlap with the CH4 bands.

Between the UV-C stage 155 and the IR stage 145 of the disinfection apparatus 10, one or more outlet fans 115b may be positioned and configured to generate a desired air flow, such as is illustrated in FIG. 10A. As may be appreciated, the one or more outlet fans 115b may be configured to generate any desired flow to suit a particular application.

### Filtration Stage

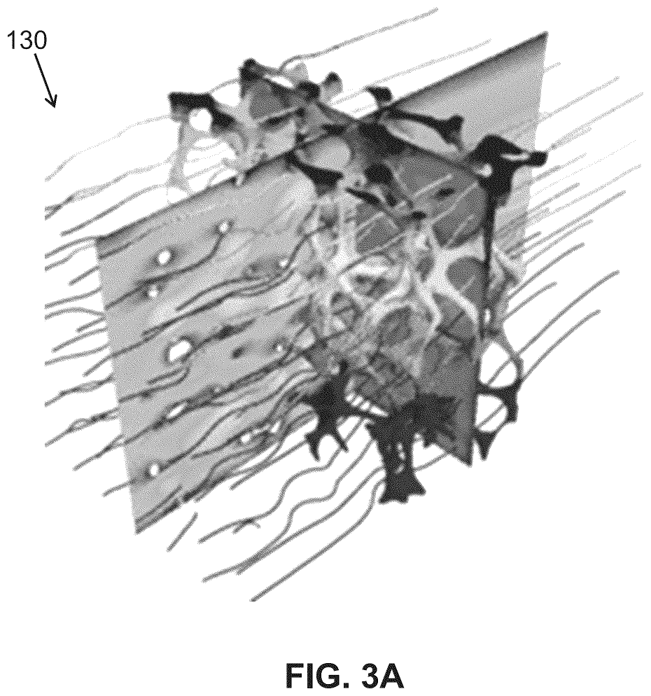

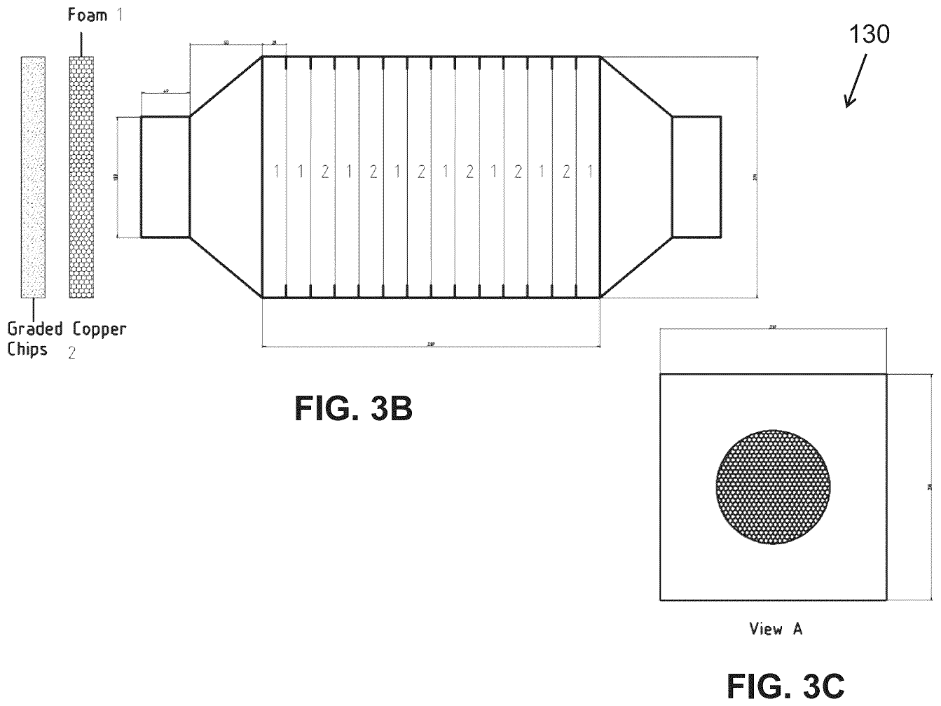

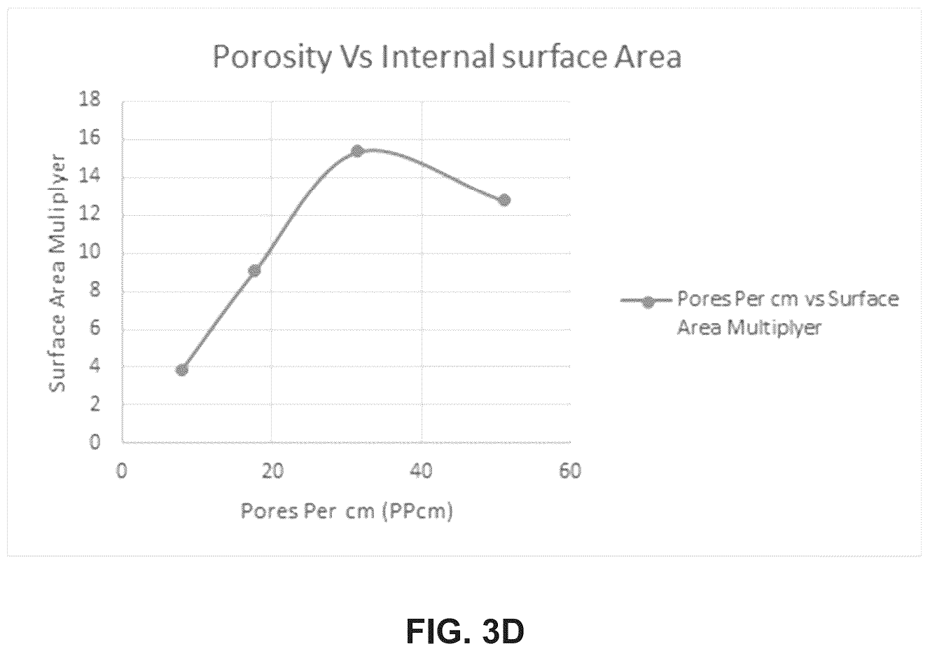

In another “stage” of exposure of the fluid flow, one or more filters (such as filters 130 illustrated in FIGS. 3A-3C) may be arranged to filter the fluid flow. As previously described, this stage of exposure may occur within the same or a separate body 100 of the disinfection apparatus 10 as any one or more of the other stages. In variations, this stage of exposure (also referred to herein as the filtration stage 135) may occur before or after either of the UV-C or IR stages 155, 145 of exposure. Generally, in the filtration stage 135, the fluid flow is filtered as the fluid flow proceeds along the tortuous flow path defined by the body 100 of the disinfection apparatus 10. The filter(s) of the filtration stage may, in variations, be configured to physically filter the fluid flow. In variations, the filter(s) of the filtration stage may be configured to filter the fluid flow through inertial impaction, interception, dissemination, dispersion, and/or diffusion. In variations, the filter(s) of the filtration stage may filter particles or other similar foreign objects from the fluid flow. The filter(s) of the filtration stage may cause thermal desiccation and/or filtration of particles or other similar foreign objects in and/or from the fluid flow.

As may be appreciated, the filter(s) of the filtration stage 135 may be of any size, shape, number, and/or type to suit a particular application. By way of non-limiting example, the filter(s) of the filtration stage may include at least one metal filter. The metal filter may, in variations, be construed of one or more antibacterial materials, such as copper, zinc, nickel, and/or combinations thereof. By way of further non-limiting example, the filter(s) of the filtration stage may be made of a metal foam. Such a metal foam filter may have a porosity of about 88% to about 95% (about 80 ppi to about 20 ppi) based on the number of holes per cm² and the depth of the filter, where the porosity is measured as void volume/total volume. By way of non-limiting example, it has been experimentally discovered, through filtration tests, that for particles in the 0.8-1.6, 0.5-0.8, and 0.1-0.4 μm size ranges, the mass-based filtration efficiency (Em) values were 99.4, 96.1, and 78.3%, respectively, for 1.0-mm-thick 15% 2ED-Cu foams, which benefited from the large surface areas of the foams. Further, for a 0.8-mm-thick 5% density 2ED-Cu foam, Em increased to 85.5% for 0.1-0.4 μm particles, while the pressure drop coefficient improved to 0.58±0.03 kPa m−1 s. For particles in the 0.5-0.8 and 0.8-1.6 μm size ranges, inertial impaction and/or interception exhibited effective filtering. For such particles in the 0.1-0.4 μm size range, diffusion/Brownian motion and/or interception exhibited effective filtering at the low and high ends of the size range, respectively. Improved efficiency in this particle size range may be achieved by increasing the foam thickness up to 2.5 mm, which was experimentally discovered to exhibit a remarkable Em of 97.0%. In one non-limiting variation, the filtration device may occupy a solid volume of about 200 cm³, a mass (if it were a solid) of 1.78 kg but actually weighing 185 g, and having about 80 pores per in (i.e., about 31 pores per cm). Such a non-limiting variation of the filtration device would have a solid volume to true volume ratio of 0.11 (i.e., a porosity of 80 ppi). In non-limiting variations, the filtration device may have a porosity of about at least 20 ppi (corresponding to a solid volume to true volume ratio of at least 0.05). In one variation, the filtration device may have a porosity of at least 45 ppi (corresponding to a solid volume to true volume ratio of at least 0.08). In one variation, the filtration device may have a porosity of at least 80 ppi (corresponding to a solid volume to true volume ratio of at least 0.11). In one variation, the filtration device may have a porosity of at least 130 ppi (corresponding to a solid volume to true volume ratio of at least 0.08). Calculated to have a surface area between 3 and 20 times the active volume of the filter, the filtration device has 3 to 20 times the equivalent flat plate surface area depending on the pore size. For example, FIG. 3D illustrates how the number of pores is affected by the surface area multiplier. It is also possible by creating a multiple layer of woven (antimicrobial metal, preferably but not limited to) copper to create the correct restriction using a copper wire with a aperture size of 1 mm up to 2 mm or alternatively 2 mm up to 3 mm with an open area of 50% up to 70% the size of the restricting filter is proportionate to the air flow.

In variations, the filter(s) may be a ridged structure, such as a ridged filter matrix. The filter(s) may, in variations, be intended to run at a constant temperature. In some variations, the temperature is at least 65° C. In some variations, the temperature is at least 70° C. In some variations, the temperature is at least 75° C. In some variations, the temperature is at least 80° C. In some variations, the temperature is at least 85° C. In some variations, the temperature is at least 90° C. In some variations, the temperature is at least 95° C. In some variations, the temperature is at least 100° C. In some variations, the temperature is at least 105° C. In some variations, the temperature is at least 110° C. In some variations, the temperature is at least 120° C. In some variations, the temperature is at least 130° C. In some variations, the temperature is at least 140° C. In some variations, the temperature is at least 150° C. In some variations, the temperature is at least 160° C. In some variations, the temperature is at least 170° C. In some variations, the temperature is at least 180° C. In some variations, the temperature is at least 190° C. In some variations, the temperature is at least 200° C. In some variations, the temperature is at least 210° C. In some variations, the temperature is at least 220° C.

In some variations, the temperature is less than or equal to 230° C. In some variations, the temperature is less than or equal to 220° C. In some variations, the temperature is less than or equal to 210° C. In some variations, the temperature is less than or equal to 200° C. In some variations, the temperature is less than or equal to 190° C. In some variations, the temperature is less than or equal to 180° C. In some variations, the temperature is less than or equal to 170° C. In some variations, the temperature is less than or equal to 160° C. In some variations, the temperature is less than or equal to 150° C. In some variations, the temperature is less than or equal to 140° C. In some variations, the temperature is less than or equal to 130° C. In some variations, the temperature is less than or equal to 120° C. In some variations, the temperature is less than or equal to 110° C. In some variations, the temperature is less than or equal to 100° C. In some variations, the temperature is less than or equal to 95° C. In some variations, the temperature is less than or equal to 90° C. In some variations, the temperature is less than or equal to 85° C. In some variations, the temperature is less than or equal to 80° C. In some variations, the temperature is less than or equal to 75° C. In some variations, the temperature is less than or equal to 70° C.

The temperature can be a minimum or maximum of any range described above.

In some variations, the temperature is within a range of about 65° C. up to 85° C. In some variations, the temperature is within a range of about 90° C. to 120° C. In some variations, the temperature is within a range of about 120° C. to 160° C. alternatively the temperature range could be 180° C. to about 300° C.

In a variation, the filter(s) may run at a temperature (e.g., a constant temperature) of about 200° C. In a variation, the filter(s) may run at a temperature (e.g., a constant temperature) of about 205° C. In a variation, the filter(s) may run at a temperature (e.g., a constant temperature) of about 210° C. In a variation, the filter(s) may run at a temperature (e.g., a constant temperature) of about 215° C. In a variation, the filter(s) may run at a temperature (e.g., a constant temperature) of about 220° C. In a variation, the filter(s) may run at a temperature (e.g., a constant temperature) of about 225° C. In a variation, the filter(s) may run at a temperature (e.g., a constant temperature) of about 230° C. By way of non-limiting example, it has been experimentally discovered that running the filter(s) at a temperature (e.g., a constant temperature) of about 222° C. led to desirable effects, namely reducing fats to basic organic compounds and deactivating protein on contact.

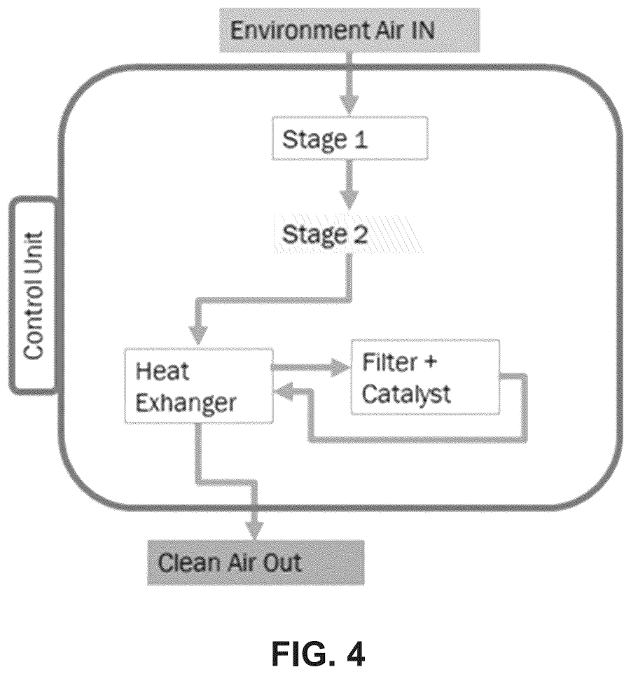

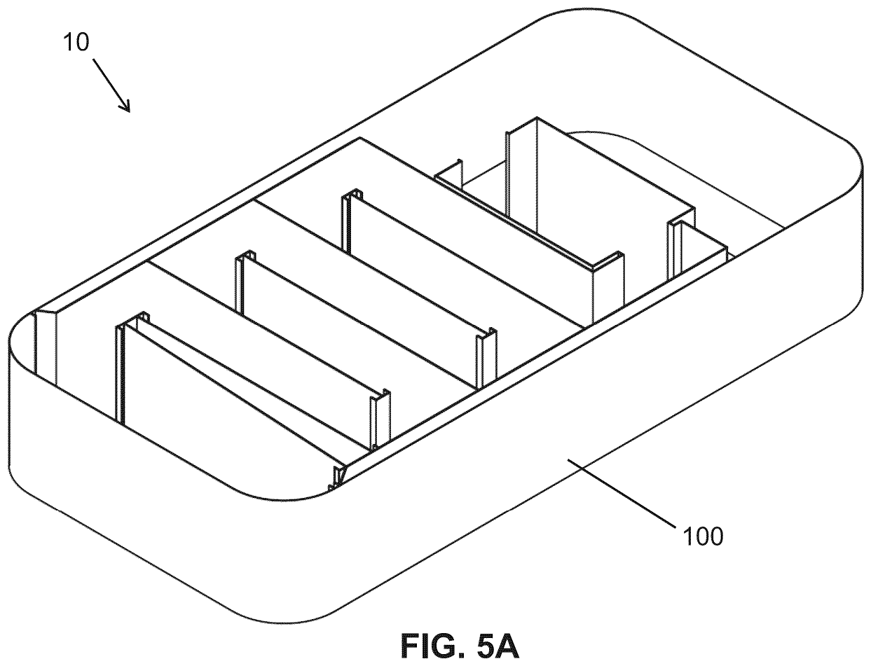

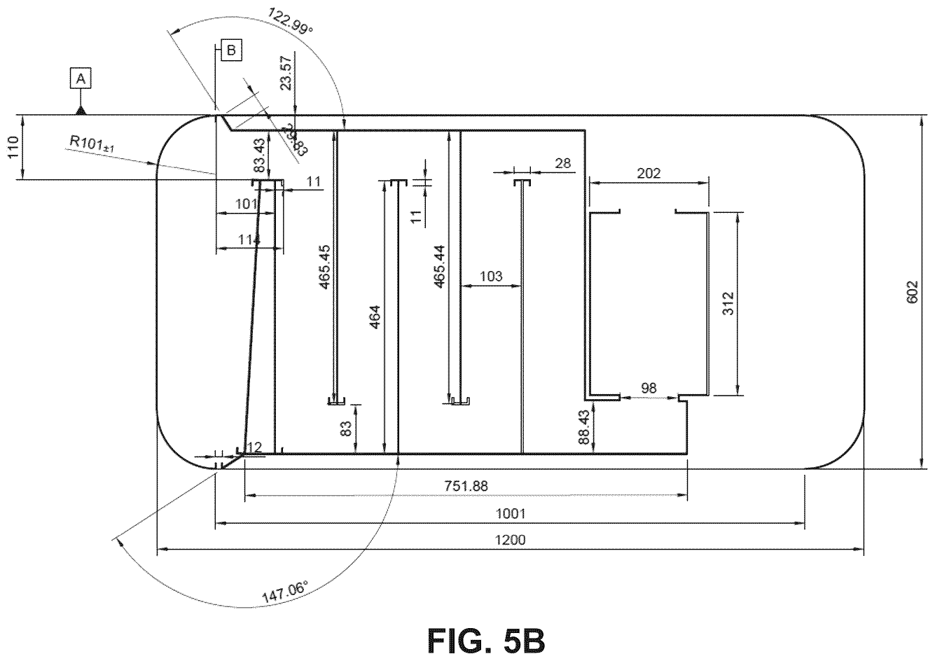

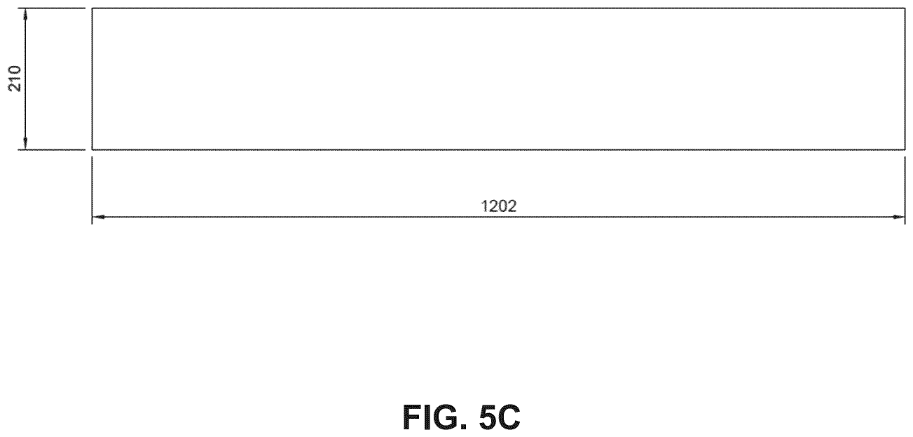

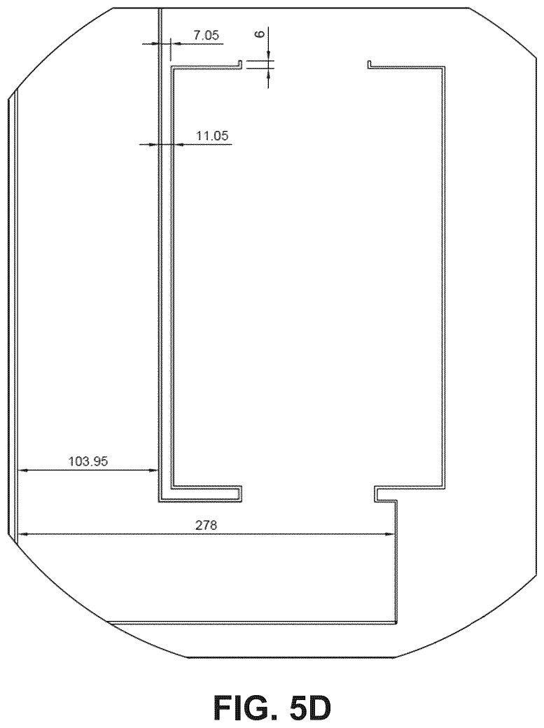

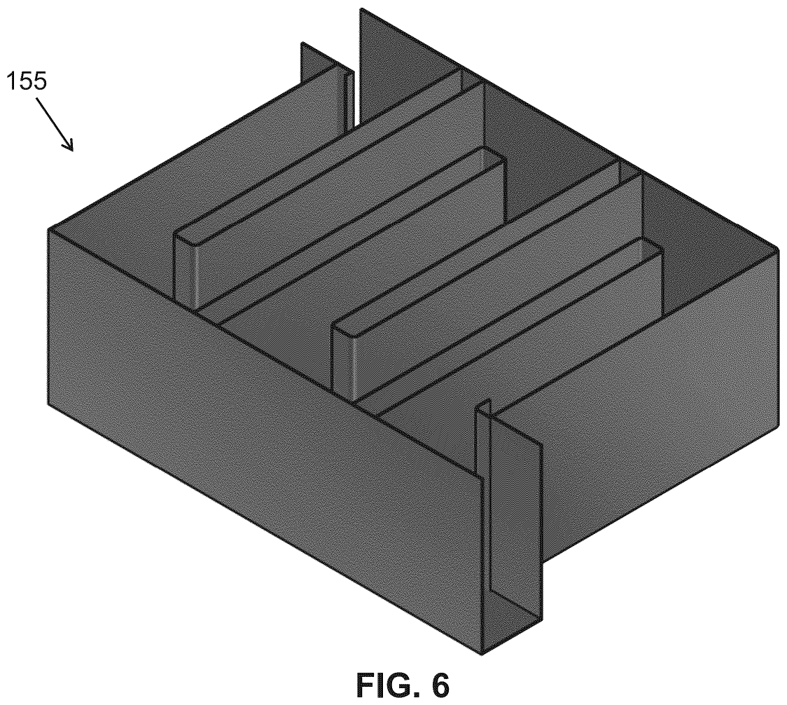

In variations, the disinfection apparatus 10 may include one or more heating elements. The heating element(s) may generally be configured to heat the fluid flow. In some variations, the heating element(s) may be arranged so as to preheat the fluid flow prior to the fluid flow passing through the filter(s). Put another way, the heating element(s) may be positioned upstream of the filter(s), such as is illustrated in FIG. 4. The heating elements(s) may, in some variations, be positioned around the filter(s). In variations, the heating element(s) may be configured to rapidly increase the fluid flow via induction heating to pass the fluid flow through the filter(s).

As may be appreciated, the heating element(s) may be of any size, shape, number, and/or type to suit a particular application. By way of non-limiting example, the heating element(s) may be configured to heat the fluid flow to a temperature of about 220° C. In a variation, the heating element(s) may be configured to heat the fluid flow such that the fluid flow has a temperature of about 180° C. to about 300° C. as the fluid flow passes through the filter(s). In a variation, the fluid flow may be heated so as to have a temperature of about 180° C. as the fluid flow passes through the filter(s). In a variation, the fluid flow may be heated so as to have a temperature of about 185° C. as the fluid flow passes through the filter(s). In a variation, the fluid flow may be heated so as to have a temperature of about 190° C. as the fluid flow passes through the filter(s). In a variation, the fluid flow may be heated so as to have a temperature of about 195° C. as the fluid flow passes through the filter(s). In a variation, the fluid flow may be heated so as to have a temperature of about 200° C. as the fluid flow passes through the filter(s). In a variation, the fluid flow may be heated so as to have a temperature of about 205° C. as the fluid flow passes through the filter(s). In a variation, the fluid flow may be heated so as to have a temperature of about 210° C. as the fluid flow passes through the filter(s). In a variation, the fluid flow may be heated so as to have a temperature of about 215° C. as the fluid flow passes through the filter(s). In a variation, the fluid flow may be heated so as to have a temperature of about 220° C. as the fluid flow passes through the filter(s). In a variation, the fluid flow may be heated so as to have a temperature of about 225° C. as the fluid flow passes through the filter(s). In a variation, the fluid flow may be heated so as to have a temperature of about 230° C. as the fluid flow passes through the filter(s). In a variation, the fluid flow may be heated so as to have a temperature of about 235° C. as the fluid flow passes through the filter(s). In a variation, the fluid flow may be heated so as to have a temperature of about 240° C. as the fluid flow passes through the filter(s). In a variation, the fluid flow may be heated so as to have a temperature of about 245° C. as the fluid flow passes through the filter(s). In a variation, the fluid flow may be heated so as to have a temperature of about 250° C. as the fluid flow passes through the filter(s). In a variation, the fluid flow may be heated so as to have a temperature of about 255° C. as the fluid flow passes through the filter(s). In a variation, the fluid flow may be heated so as to have a temperature of about 260° C. as the fluid flow passes through the filter(s). In a variation, the fluid flow may be heated so as to have a temperature of about 265° C. as the fluid flow passes through the filter(s). In a variation, the fluid flow may be heated so as to have a temperature of about 270° C. as the fluid flow passes through the filter(s). In a variation, the fluid flow may be heated so as to have a temperature of about 275° C. as the fluid flow passes through the filter(s). In a variation, the fluid flow may be heated so as to have a temperature of about 280° C. as the fluid flow passes through the filter(s). As previously described, by way of non-limiting example, it has been experimentally discovered that preheating the fluid flow (e.g., upstream of the filter(s)) to a temperature of about 222° C. led to desirable effects, namely reducing fats to basic organic compounds and deactivating protein on contact. In variations, the heating element(s) may be one or more thermal heating elements.

In variations, a heat exchanger may be employed, such as is illustrated in FIG. 4. The heat exchanger may, in variations, be positioned downstream of the IR stage 145, upstream of the filtration stage 135, across the inlet and/or outlet of the filtration stage 135, and/or downstream of the filtration stage 135. The heat exchanger may be configured in a heat recovery configuration to reduce the energy requirements of the IR emitter(s) of the IR stage and/or to reduce the air temperature at the outlet(s) 120 of the disinfection apparatus 10. In variations employing a heat exchanger, fluid flow into the heat exchanger from the IR stage after exposure to the IR emitter(s) may experience a temperature rise at an inlet bank of the heat exchanger, thereby delivering higher temperature fluid into the filter(s), where additional heat may be added. The fluid flow may flow from the exit of the filtration stage into the heat exchanger, delivering cooled fluid from to the exit bank of the heat exchanger. In variations, an additional thermal sink may be employed to aid in further heat dissipation.

In variations, a control system or controller may be employed, such as is illustrated in FIG. 4. The controller may, in variations, provide for automated or remote temperature modulation and/or air quality monitoring. In variations, the foregoing can be in communication and/or connected with a computer interface or the like and/or stored in a cloud-based system. Through operation of the controller, the disinfection apparatus may additionally be capable of heating and/or cooling the expelled fluid flow pursuant to a given set of parameters. Air quality monitoring could also be employed to validate total sanitation and/or provide reassurance of minimalized risk of infection. The control system or controller may provide one or more of the following functions: air monitoring; power management; thermal management; and data management and/or transfer.

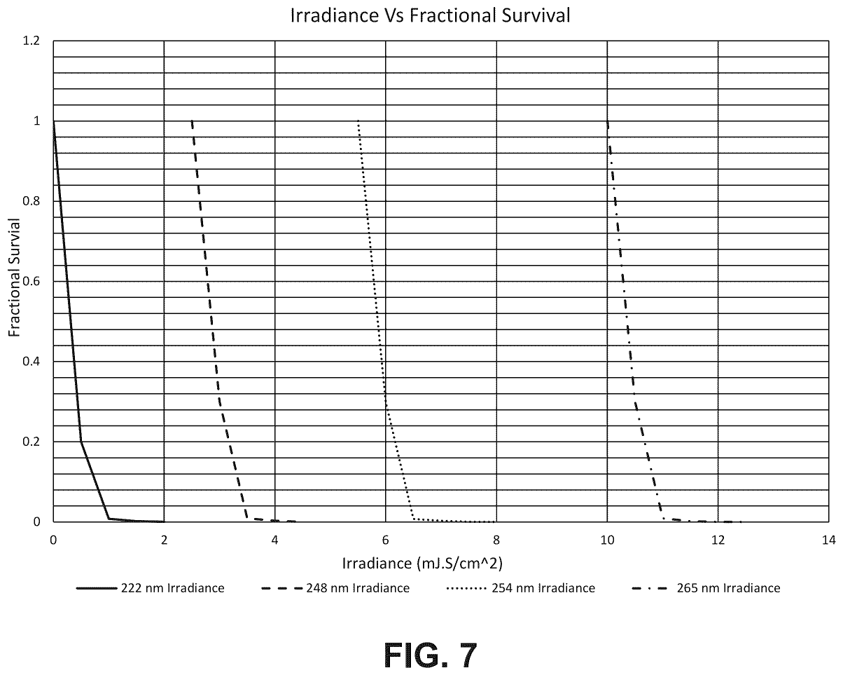

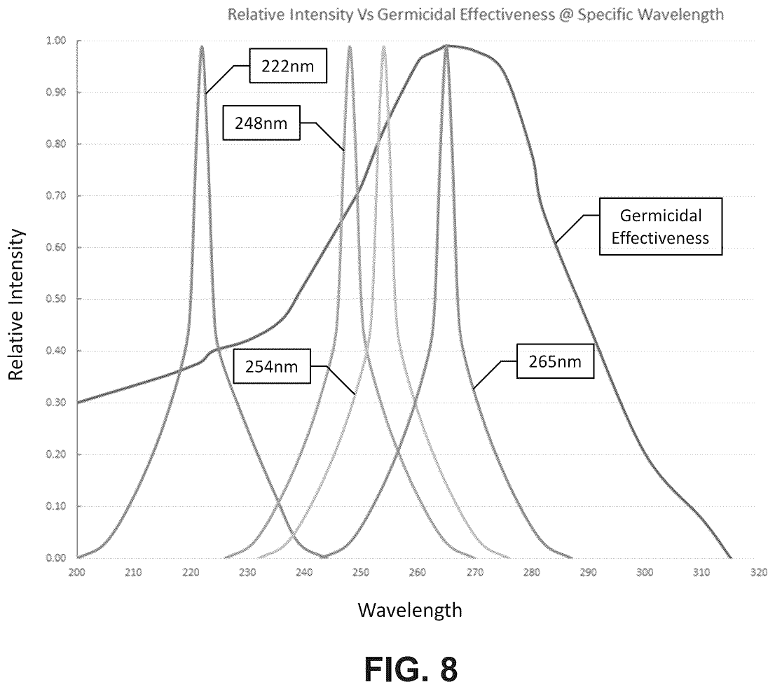

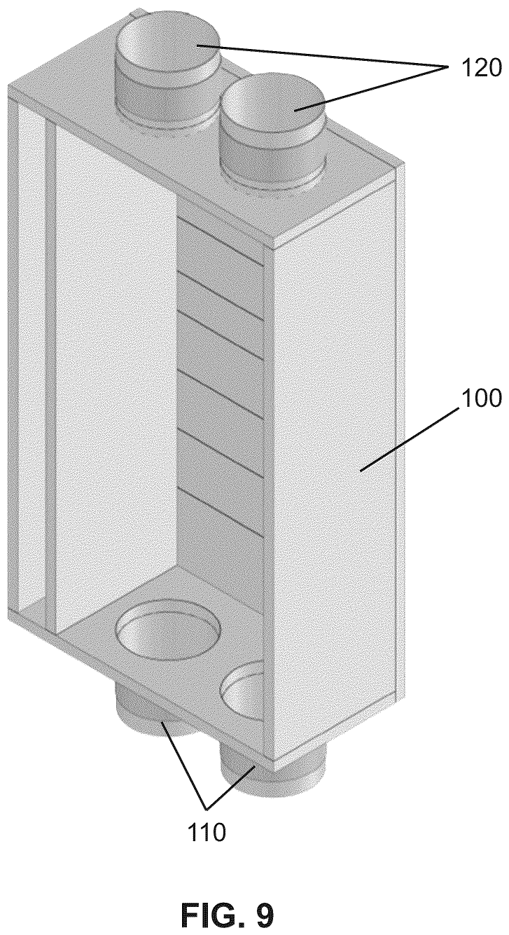

FIG. 7 illustrates the effectiveness of the non-conventional dosage of light at a wavelength of 222 nm, which, as shown, may demonstrate strong disinfecting and/or sanitization effects without presenting significant risk to mammalian cells. As also shown in FIG. 8, germicidal effects may occur at specific wavelengths.

As described above, the control system or controller may provide air monitoring functions, such that parameters of the fluid flow may be monitored at the inlet and/or the outlet of the disinfection apparatus. Such parameters may include, but are not limited to, temperature, pressure, velocity, MFO content, ozone, odour, room CO and CO₂, and occupancy levels.

As described above, the control system or controller may provide power management functions (e.g., by utilizing an electronic control unit or ECU), including, but not limited to, the use of real-time monitoring and/or differential calculations to optimize the power requirements and/or power utilisation of the disinfection apparatus based on maintaining a “clean” room.

As described above, the control system or controller may provide thermal management functions (e.g., by utilizing an electronic control unit or ECU), including, but not limited to, maintaining a stable operating temperature for the disinfection apparatus, such as through control of one or more cooling fans for key modular equipment.

As described above, the control system or controller may provide data management and/or transfer functions, including, but not limited to, gathering and/or utilizing real-time data to optimize the disinfection apparatus, such as by providing secondary fault monitoring.

In addition to or alternatively to the foregoing, in variations, power supply stability may be provided to ensure realization of LED life. In variations, each unit may be linked to RFID tags of the key equipment in each module. The disinfection apparatus may, in variations, include primary and secondary battery power, such as supplied via a frequency modulator, and/or the inlet power may be adjusted for local power requirements. In variations, solar recovery from the emitter(s) (including the UV and/or IR emitters) may be provided, which may allow for a percentage of power for recirculation within the disinfection apparatus.

Any of the following variations, or any portions thereof, can be combined with any of the following in any variation.

The disclosure provides for disinfection apparatuses or fixed UV radiation devices for the purpose of disinfecting a fluid flow (e.g., an air flow). In variations, UV light may be employed to disinfect the fluid flow, infrared light may be employed to sterilize the fluid flow, and a metal foam filtration device may be employed (and, in some variations, heated) to provide physical filtration. The filtration device may, in certain variations of copper and copper zinc alloy. In variations, the filtration device may remove biofilm and detritus from a fluid flow, thereby removing the material synonymous with assisting the transmission of viral and biological infectious microorganisms.

In a variation, the disinfection apparatus may include any one or more of the following aspects: an array housing having a substantially tortuous planar array with a number of 90° bends and a flat smooth surface with opposed LED emitters and/or incandescent lamps at an emittance of 230 nm to 278 nm, or any other range as described previously. A plurality of UV-C emitters coupled to the 90° bends and planar array surface in a substantially horizontally configuration in relation to each other; at least one UV sensor coupled to the substantially planar array surface; at least one orientation sensor coupled to an array inlet; a base housing defining an interior portion; a number of motors to drive air through a tortuous flow path being housed in the interior portion of the base housing. In variations, it may be advantageous to include an array of solar collectors to minimize energy waste, a battery pack being housed in the base housing and engaging with automated software to provide real-time data and warn of any high contamination loads or power loss or system failure. In variations, the controller, the emitters, and/or the UV sensor may also run off a backup circuit as required, for example, if the variation is to be used by military or law enforcement or where a power failure may need immediate attention.

In a variation, a method for fluid flow disinfection may use UV-C and IR radiation and may include one or more of the following aspects: delivering, with an array of UV-C emitters, a beam of UV in excimer form of radiation to a first chamber of a device; measuring, with at least one UV-C and/or IR sensor, an amount of UV energy reflected from the first chamber of the device; measuring, with a processor, a UV energy threshold for the least one UV-C and/or IR sensor; in response to satisfying a UV and IR energy threshold received by the at least one UV-C and/or IR sensor, delivering, to a second chamber of the device, a multiple wavelength beam of UV radiation; and operating a heating element placed around a metal filter in response to satisfying an energy threshold required to heat and maintain a constant temperature across the entire surface of the filter.

In yet another variation, a system for air disinfection using UV and IR radiation includes one or more of the following aspects: at least one fixed UV, IR, and temperature disinfection apparatus, which can be added to an existing HVAC system, with the UV, IR, and temperature disinfection apparatus including an array housing having a substantially constricted or tortuous path defined by a number of 90° bends (e.g., more than two bends) and a planar array surface; a plurality of UV and IR emitters coupled to the substantially planar array surface, with constricted apertures, the UV and IR emitters being coupled horizontally or adjacent to the substantially planar array surface in a substantially horizontal configuration in relation to each other; at least one UV and IR sensor coupled to the substantially planar array surface; at least one orientation sensor coupled to the array inlet housing; and a base housing defining an interior portion.

In a variation, a germicidal disinfection apparatus includes one or more of the following aspects: a housing assembly comprising a base housing and an array housing; a forced air motor or plural motors being housed within the device; a plurality of emitters including at least one first emitter configured to emit ultraviolet light at a wavelength between 130 nm to 280 nm and emitters delivering IR radiation at 1400 nm to 1500 nm as has been shown to be advantageous for H₂O absorption; at least one second emitter configured to emit visible light at a wavelength between 380 nm and 405 nm; a controller being operably engaged with the range of emitters and a heating element to modulate a duty cycle, with the controller including at least one processor and computer-read writable instructions and diagnostics stored thereon that, when executed, cause the processor to perform one or more operations, the one or more operations including modulating a duty cycle monitoring and controlling air temperature and confirming air quality at the outlet of the apparatus.

In a variation, the germicidal disinfection apparatus may be designed to work in conjunction with a functioning cloud-based temperature control system that does not have conditioned air. The controller may be further configured to execute one or more operations including calculating a radiation dose delivered by the emitters where air flow may increase or decrease according to environmental occurrence.

In a variation, the germicidal disinfection apparatus may include at least one ranging sensor coupled to a surface of the housing assembly and in communication with the controller.

In a variation, a method may include controlling microorganisms in an interior environment by positioning a germicidal disinfection apparatus in a location of the interior environment.

In a variation, a germicidal disinfection apparatus may include one or more of the following aspects: a housing assembly comprising a base housing and an array housing; a collection of air forcing motors being housed in the housing; a plurality of emitters comprising an array of LEDs having a beam angle of less than or equal to 180 degrees, the plurality of emitters including at least one first emitter configured to emit ultraviolet light at a wavelength between 130 nm to 280 nm, at least one second emitter configured to emit visible light at a wavelength between 380 nm and 405 nm, a third emitter configured to emit light at a wave length of between 1400 nm and 1500 nm, and an array of emitters configured to emit visible light at a wavelength between 400 nm and 600 nm; and a controller in communication with the plurality of emitters to modulate the required dose identified by environmental parameters.

In a variation, a mode of operation of the germicidal disinfection apparatus may include one or more operations for modulation according to a kinetic model including an effective radiation kill dose for at least one bacteria, virus, or fungus. In variations, a mode of operation of the germicidal disinfection apparatus may include one or more operations for modulating a pulse width of the at least one first emitter and the at least one second emitter according to a kinetic requirement comprising an effective radiation kill dose for at least one bacteria, virus, or fungus.

As will be appreciated by those skilled in the art, the disinfection apparatuses of the disclosure may be used in a variety of applications. By way of non-limiting example, it is contemplated that the disinfection apparatuses described herein may be incorporated into larger systems or structures, such as theatres, shopping malls, retail shops, airports, train stations, hospital operating rooms, dentist and orthodontic practices, vehicles and/or passenger transport vessels (e.g., ships, trains, trams, aircraft), sealed rooms, and/or forced air systems. Further, the disinfection apparatuses described herein may be used in any environment in which may be desirable to achieved disinfection of a fluid flow.

### Example

Electrophoresis using Deltadot Technology was used to confirm peak absorption of specific infrared wavelengths of post-adsorbed bacteria. Cellular morbidity was confirmed by through cell wall destruction and phospholipid absorption. Using the Beer-Lambert law it was possible to relate the amount of light absorbed to the concentration of the absorbing molecule. At a wavelength of 260 nm, the average extinction coefficient for double-stranded DNA was determined to be 0.020 (μg/ml)−1 cm⁻¹. For single-stranded DNA, the average extinction coefficient was 0.027 (μg/ml)−1 cm⁻¹. For single-stranded RNA, the average extinction coefficient was 0.025 (μg/ml)−1 cm⁻¹. For short single-stranded oligonucleotides, the average extinction coefficient was dependent on the length and base composition. Thus, an Absorbance (A) of 1 corresponded to a concentration of 50 μg/ml for double-stranded DNA.

A suspended channel resonator was embedded inside a microbeam to overcome the limitations of liquid damping and achieve higher mass resolution. The beam was excited into resonance in a vacuum for increased mass resolution and higher reproducibility. To obtain three orthogonal signals-adsorbed mass, adsorption stress and mid-infrared spectroscopy of the adsorbates. Beam resonance frequency, resulting from changes in the inertial mass of the aerosol-filled Beam. Adsorption-induced stress originates from illuminating the Beam with infrared radiation. Absorption of specific infrared wavelengths by the adsorbed bacteria causes additional Beam deflection because of non-radiative decay. The nanomechanical bending of the Beam, as a function of illuminating wavelength, resembles the infrared absorption spectrum of the bacteria. Sensitive monitoring of this fluctuation allows the sensor to discriminate between intact and dead Escherichia coli (E. coli), as well as characterize the metabolic response of E. coli to antibiotics.

To demonstrate bacterial destruction, we used L. monocytogenes, a serious food-borne pathogen that has a mortality rate exceeding 20%. Before bacterial injection into the sensor (102 cells in 100 μl), the inner surface of the chip was functionalized with the anti-L. monocytogenes monoclonal antibody.

This mechanical infrared absorption of the bacteria displays a typical spectrum with a distinct absorption peak at 1,451 cm⁻¹, suggesting a peptidoglycan layer of the bacterial cell wall (FIG. 2c). Absorption bands observed at 1,233 and 1,213 cm⁻¹ (FIG. 2c) are due to the C—O—C ester and P═O vibrations of the bacteria phosphate diester groups, respectively. Two other vibrational bands also appear during irradiation of the sensor with higher wavelengths, indicating a P—OH (1,100 cm⁻¹) and polysaccharide group (1,023 cm⁻¹) in the bacterial cell wall. These observed infrared absorption bands are a characteristic fingerprint of the bacteria.

### Selectivity

These variations can be attributed to the asymmetric stretching of P═O in the phosphodiester backbone of nucleic acids (at ˜1,213 cm⁻¹), the asymmetry of the peptidoglycan layer of the bacterial cell wall (at 1,451 cm⁻¹) and the lipid groups (between 1,000 and 1,023 cm⁻¹) in the bacterial cell wall.

### Live Versus Dead Bacteria

To investigate whether bacteria have been killed or placed in a dormancy state. As expected, the bacteria exposed to IR at a dose of 3. Σ mJ/s/cm\hat{ }3 resulted in permanent damage and the viability of the microorganism resulted in IR morbidity.

TABLE 1

| | | | |
|---|---|---|---|
| Edehyd | Energy for dehydration | mJ/s/cm\hat{ }3 | 3.E+01 |
| Energy required | = (Area × Edehyd × 100000) | mJ/s/cm\hat{ }3 | 4.E+04 |
| for a given VFR | | | |
| Energy developed | = [(Area × (Length*1000000)) × | mJ/s/cm\hat{ }3 | 1.E+05 |
| | LED power output] | | |
| Irradiance | Energy dev. X Rt | mJ/s/cm\hat{ }3 | 4.E+04 |

| | |
|---|---|
| Irradiance/Energy Required = | 0.99 |
| Calculated Reserve factor | |

The particular variations disclosed above are illustrative only, as the variations may be modified and practiced in different but equivalent manners apparent to those skilled in the art having the benefit of the teachings herein. It is therefore evident that the particular variations disclosed above may be altered or modified, and all such variations are considered within the scope and spirit of the application. Accordingly, the protection sought herein is as set forth in the description. Although the present variations are shown above, they are not limited to just these variations, but are amenable to various changes and modifications without departing from the spirit thereof. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed variations teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A disinfection apparatus, comprising:
a body defining at least one inlet and at least one outlet;
one or more inlet fans proximate the at least one inlet and configured to generate a fluid flow in a direction toward the at least one outlet;
a first stage fluidly associated with the at least one inlet, the first stage defining a first tortuous flow path delineating at least two turns such that the fluid flow changes direction at least twice as the fluid flow proceeds through the first tortuous flow path, the first stage comprising one or more emitters configured to emit ultraviolet light at one or more wavelengths upon the fluid flow;
a second stage fluidly associated with the first stage, the second stage comprising one or more emitters configured to emit infrared light at one or more wavelengths upon the fluid flow; and
a third stage fluidly associated with the second stage, the third stage comprising one or more metal filters constructed of one or more antibacterial metals and configured to filter the fluid flow;
the at least one outlet fluidly associated with the third stage.

2. The disinfection apparatus according to claim 1, wherein the one or more emitters of the first stage are configured to emit ultraviolet light at at least one wavelength from 130 nm to about 405 nm upon the fluid flow.

3. The disinfection apparatus according to claim 1, wherein the one or more emitters of the second stage are configured to emit infrared light at a first wavelength of the at least one wavelength from 1200 nm to 1510 nm upon the fluid flow.

4. The disinfection apparatus according to claim 1, wherein the one or more emitters of the second stage are configured to emit infrared light at a second wavelength of the at least one wavelength from 1380 nm to 1800 nm upon the fluid flow.

5. The disinfection apparatus according to claim 1, wherein at least one metal filter of the one or more metal filters in the third stage is copper or copper alloy.

6. The disinfection apparatus according to claim 1, further comprising one or more outlet fans between the first stage and the second stage and configured to force the fluid flow in a direction toward the at least one outlet.

7. The disinfection apparatus according to claim 1, wherein the second stage defines a second tortuous flow path delineating at least two turns such that the fluid flow changes direction at least twice as the fluid flow proceeds through the second tortuous flow path.

8. The disinfection apparatus according to claim 1, wherein the first tortuous flow path is defined by a plurality of spaced-apart walls, each of the plurality of spaced-apart walls spanning less than a full dimension of the first stage of the body such that the fluid flow proceeds around ends of each of the plurality of spaced-apart walls.

9. The disinfection apparatus according to claim 1, wherein the first tortuous flow path is defined by a plurality of spaced-apart walls, each of the plurality of spaced-apart walls spanning a full dimension of the first stage of the body and defining an aperture configured to permit the fluid flow to pass therethrough.

10. The disinfection apparatus of claim 9, wherein each aperture is semicircular and configured to create or increase turbulence and/or pressure of the fluid flow within the first stage.

11-25. (canceled)

26. The disinfection apparatus of claim 1, wherein the first tortuous flow path is a substantially serpentine flow path.

27. The disinfection apparatus of claim 1, further comprising one or more motors configured to force the fluid flow through the disinfection apparatus.

28. The disinfection apparatus of claim 1, further comprising a heating element configured to heat the fluid flow.

29. The disinfection apparatus of claim 28, wherein the heating element is positioned upstream of the one or more metal filters or around the one or more metal filters.

30. The disinfection apparatus of claim 28, wherein the heating element is configured to heat the fluid flow such that the fluid flow has a temperature of about 180° C. to about 300° C. as the fluid flow passes through the one or more metal filters.

31. A disinfection apparatus for imparting electromagnetic energy upon a fluid flow, the disinfection apparatus comprising:
a body defining a tortuous flow path delineating at least two turns such that the fluid flow changes direction at least twice as the fluid flow proceeds through the tortuous flow path; and
a plurality of emitters within the body and configured to emit ultraviolet light or infrared light or both upon the fluid flow as the fluid flow proceeds through the tortuous flow path.

32. A method of imparting electromagnetic energy upon a fluid flow for disinfection thereof, the method comprising:
flowing a fluid flow through a tortuous flow path delineating at least two turns such that the fluid flow changes direction at least twice as the fluid flow proceeds through the tortuous flow path; and
emitting ultraviolet light or infrared light or both upon the fluid flow as the fluid flow proceeds through the tortuous flow path.

33. The disinfection apparatus according to claim 1, wherein, in the second stage, a relative humidity of the fluid flow at a constant temperature and a constant pressure is reduced by at least 10%.

34. The disinfection apparatus according to claim 33, wherein the relative humidity of the fluid flow at the constant temperature and the constant pressure is reduced by 10%-45%.

35. The disinfection apparatus according to claim 33, wherein the relative humidity of the fluid flow at the constant temperature and the constant pressure is reduced by 10%-35%.
